(19) United States
(12) Patent Application Publication
(10) Pub. No.: US 2009/0043240 A1
(43) Pub. Date: Feb. 12, 2009

(54) Method and apparatus for blood transport using a pressure controller in measurement of blood characteristics

(76) Inventors: Mark Ries Robinson, Albuquerque, NM (US); John O'Mahony, Maple Grove, MN (US)

Correspondence Address: V. Gerald Grafe, esq., P.O. Box 2689, Corrales, NM, 87048, US

(21) Appl. No.: 12/108,250

(22) Filed: Apr. 23, 2008

Related U.S. Application Data
(63) Continuation-in-part of application No. PCT/US2006/060850, filed on Nov. 13, 2006.
(60) Provisional application No. 60/913,582, filed on Apr. 24, 2007.
(60) Provisional application No. 60/737,254, filed on Nov. 15, 2005.
(60) Provisional application No. 60/791,719, filed on Apr. 12, 2006.

(51) Int. Cl. A61M 1/34

(52) U.S. Cl. 604/6.11

(57) ABSTRACT

A method and app for controlling blood withdrawal and infusion flow rate with the use of a pressure controller in connection with a blood measurement system. The pressure controller uses pressure targets based upon occlusion limits that are calculated as a function of flow. The controller has the ability to switch from controlling withdrawal pressure to controlling infusion pressure based upon the detection of an occlusion. The controller distinguishes between partial and total occlusions of the withdrawal vein providing blood access. Depending on the nature of occlusion, the controller limits or temporarily reverses blood flow and, thus, prevents withdrawal vessel collapse or reverses blood flow to quickly infuse blood into the vessel without participation from operator.

### CROSS-REFERENCE TO RELATED APPLICATIONS

This application claims priority to U.S. provisional application 60/913,582, filed Apr. 24, 2007; and is a continuation-in-part of international (PCT) application PCT/US2006/060850 filed Nov. 13, 2006, which claimed priority to U.S. provisional application 60/737,254 filed Nov. 15, 2005, and which claimed priority to U.S. provisional application 60/791,719 filed on Apr. 12, 2006. This application is related to U.S. patent application Ser. No. 11/154,569 filed Jun. 17, 2005; which is a continuation of U.S. patent application Ser. No. 10/601,574 filed Jun. 24, 2003, which is a divisional application of U.S. patent application Ser. No. 09/703,702 filed Nov. 2, 2000, now U.S. Pat. No. 6,585,675. Each of the aforementioned applications is incorporated by reference herein.

### FIELD OF THE INVENTION

The invention relates to the field of pressure controllers for fluidic pumping systems and, in particular, to pressure controllers for intravenous blood pumps used in connection with systems that measure blood characteristics.

### BACKGROUND OF THE INVENTION

More than 20 peer-reviewed publications have demonstrated that tight control of blood glucose significantly improves critical care patient outcomes. Tight glycemic control (TGC) has been shown to reduce surgical site infections by 60% in cardiothoracic surgery patients and reduce overall ICU mortality by 40% with significant reductions in ICU morbidity and length of stay. See, e.g., Furnary Tony, Oral presentation at 2005 ADA annual, session titled “Management of the Hospitalized Hyperglycemic Patient;” Van den Berghe et al., NEJM 2001; 345:1359. Historically, caregivers have treated hyperglycemia (high blood glucose) only when glucose levels exceeded 220 mg/dl. Based upon recent clinical findings, however, experts now recommend IV insulin administration to control blood glucose to within the normoglycemic range (80-110 mg/di). Adherence to such strict glucose control regimens can require near-continuous monitoring of blood glucose and frequent adjustment of insulin infusion to achieve normoglycemia while avoiding risk of hypoglycemia (low blood glucose). In response to the demonstrated clinical benefit, approximately 50% of US hospitals have adopted some form of tight glycemic control with an additional 23% expected to adopt protocols within the next 12 months. Furthermore, 36% of hospitals already using glycemic management protocols in their ICUs plan to expand the practice to other units and 40% of hospitals that have near-term plans to adopt TGC protocols in the ICU also plan to do so in other areas of the hospital.

Given the evidence for improved clinical outcomes associated with tight glycemic control, hospitals are under pressure to implement TGC as the standard of practice for critical care and cardiac surgery patients. Clinicians and caregivers have developed TGC protocols that use IV insulin administration to maintain normal patient glucose levels. To be safe and effective, these protocols require frequent blood glucose monitoring. Currently, these protocols involve periodic removal of blood samples by nursing staff and testing on handheld meters or blood gas analyzers. Although hospitals are responding to the identified clinical need, adoption has been difficult with current technology due to two principal reasons: fear of hypoglycemia, and burdensome procedures.

Fear of hypoglycemia. The target glucose range of 80-110 mg/dl brings the patient near clinical hypoglycemia (blood glucose less than 50 mg/dl). Patients exposed to hypoglycemia for greater than 30 minutes have significant risk of neurological damage. IV insulin administration with only intermittent glucose monitoring (typically hourly by most TGC protocols) exposes patients to increased risk of hypoglycemia. A recent letter to the editors of Intensive Care medicine noted that 42% of patients treated with a TGC protocol in the UK experienced at least one episode of hypoglycemia. See, e.g., lain Mackenzie et al, “Tight glycaemic control:a survey of intensive care practice in large English Hospitals;” Intensive Care Med (2005) 31:1136. In addition, handheld meters require procedural steps that are often cited as a source of measurement error, further exacerbating the fear (and risk) of accidentally taking the blood glucose level too low. See, e.g., Bedside Glucose Testing systems, CAP today, April 2005, page 44.

Burdensome procedure. Most glycemic control protocols require frequent glucose monitoring and insulin adjustment at 30 minute to 2 hour intervals (typically hourly) to achieve normoglycemia. Caregivers recognize that glucose control would be improved with continuous or near-continuous monitoring. Unfortunately, existing glucose monitoring technology is incompatible with the need to obtain frequent measurements. Using current technology, each measurement requires removal of a blood sample, performance of the blood glucose test, evaluation of the result, determination of the correct therapeutic action, and finally adjustment to the insulin infusion rate. High measurement frequency requirements coupled with a labor-intensive and time-consuming test places significant strain on limited ICU nursing resources that already struggle to meet patient care needs.

As used herein, “measuring a characteristic of blood” or similar phrases include any determination of a property or characteristic of blood. Examples include measurement of analyte presence or concentration in blood, such as measurement of the concentration of glucose, glucose derivatives, therapeutic agents, or other analytes present in blood. Other examples include the determination of pH, hematocrit, hemoglobin, cholesterol, albumin, total protein, carbon dioxide, oxygen saturation, bicarbonate, water concentration, and plasma volume.

Characteristics of blood are often measured by withdrawing a blood sample, transporting the sample to a remote analyzer, and determining the characteristic with the analyzer. The sample is generally consumed or destroyed in the process. The attendant blood loss can be undesirable if frequent determinations are desired (such as for control of glucose), and with patients where minimal blood loss is important (such as many surgical settings). An automated system that withdraws blood, determines the characteristic, and then returns some or all of the withdrawn blood can allow such determinations without the undesirable blood loss.

While the blood is outside of the body, it flows through an “extracorporeal blood circuit” of tubes, filters, pumps and/or other medical components. In some treatments, the blood flow is propelled by the patient's blood pressure and by gravity, and no artificial pump is required. In other treatments, blood pumps in the extracorporeal circuit provide additional force to move the blood through the circuit and to control the flow rate of blood through the circuit. These pumps can be peristaltic or roller pumps, which are easy to sterilize, are known to cause minimal clotting and damage to the blood cells, and are inexpensive and reliable. It is recognized that other pumps may be applicable for use with a blood circuit.

Brushed and brushless DC motors are commonly used to rotate peristaltic pumps. A motor controller regulates the rotational speed of blood pumps. The speed of a pump, often expressed as rotations per minute (RPM), regulates the flow rate of the blood through the circuit. Each revolution of the pump moves a known volume of blood through the circuit. Thus, the blood flow rate through the circuit can be derived from the pump speed. Accordingly, the pump speed provides a relatively accurate indicator for the volume flow of blood through an extracorporeal circuit.

Existing pump controllers can be as simple as a potentiometer that regulates the voltage to the pump DC motor. The pump speed is proportional to the voltage applied to the pump motor. By increasing the voltage, the speed of the pump increases and consequently the blood flow increases through the extracorporeal circuit. More sophisticated existing pump controllers, such as used in contemporary dialysis machines, include a microprocessor that executes a software/firmware program to regulate the pump speed and thus blood flow in accordance with pump/flow settings entered by an operator. In these controllers, the micro-processor receives input commands from an operator who selects a desired blood flow using a user interface on the controller housing. The microprocessor determines the pump motor speed needed to provide the selected blood flow rate, and then issues commands to the pump motor to run at the proper speed.

To improve the accuracy and reliability of the blood flow through an extracorporeal circuit, existing pump controller microprocessors receive feedback signals from, for example, tachometers or optical encoders that sense the actual speed of the pump motor. Similarly, feedback signals have been provided by ultrasonic flow probes that measure the actual flow of blood in the extracorporeal circuit. By comparing the desired pump speed or flow rate to the measured pump speed or flow rate, the microprocessor can properly adjust the pump speed to correct for any difference between the desired and actual speed or rate. In addition, a calculated or measured flow value (actual flow rate) can be displayed on the pump console for viewing by the operator for a visual comparison with the desired flow setting.

The microprocessor controllers for blood pumps have, in the past, relied on both open and closed loop control of the motor speed. The open loop control normally consists of a constant feed forward voltage (based on the back EMF constant of the motor). The closed loop control systems use velocity feedback in the form of a tachometer, encoder or resolver to maintain constant pump flow. A constant flow control loop allows a user to set the blood flow rate, and the controller regulates the pump speed to maintain a constant blood flow, unless a malfunction occurs that would require the pump to be shut-down to protect the patient. The open loop control systems have the disadvantage that an increase or decrease in torque or motor resistance due to temperature will result in some variation in pump flow. This variation is generally small when the motor is geared. Torque variations are less of a concern in closed loop control systems because such systems use the motor velocity as feedback and can adjust the supply current or voltage to the motor in order to maintain constant velocity.

Existing blood pump controllers include various alarms and interlocks that can be set by a nurse or a medical technician (either of which is an “operator” of the controller), and are intended to protect the patient. In a typical dialysis apparatus, the blood withdrawal and blood return pressures are measured in real time, so that sudden pressure changes can be quickly detected. Sudden pressure changes in the blood circuit are treated as indicating an occlusion or a disconnect in the circuit. The detection of a sudden pressure change causes the controller to stop the pump and cease withdrawal of blood. The nurse or operator can set the alarm limits for the real time pressure measurements well beyond the expected normal operating pressure for the selected blood flow, but within a safe pressure operating range.

Examples of existing blood pump controllers are disclosed in U.S. Pat. Nos. 5,536,237 ('237 patent) and 4,657,529 ('529 patent). The controllers disclosed in these patents purport to optimize the rate of blood flow through a blood circuit based on a pressure vs. flow rate control curve. However, these patents do not teach controlling a blood pump based on control curves for both withdrawal and infusion pressures, and do not suggest reversing blood flow to relieve an occlusion. The authors of the '237 and '529 patents acknowledged that during blood withdrawal, treatment is often interrupted if the vein is collapsed. They further acknowledged that as the result of such collapse the needle could be drawn into the blood vessel wall that makes the recovery difficult. The remedy proposed by these authors is to always withdraw blood at a rate that prevents the collapse of the vein by applying a complex system of identification of the vein capacity prior to treatment. The '237 patent specifically addresses the difficulty of generating ad-hoc pressure flow relationships for individual patients with low venous flow capacity. However, the experience of the present applicants is that the withdrawal properties of venous access in many patients are prone to frequent and often abrupt changes during treatment. Accordingly, the approach advanced in the '237 and '529 patents of attempting to always avoid vein collapse will fail when a vein collapse condition occurs and does not provide any remedy for vein collapse (other than to terminate treatment and call a nurse or doctor).

Pressure conditions in a blood circuit can change because of blood viscosity changes (that in turn affect flow resistance), and because of small blood clots that form where blood stagnates on the surface of tubes and cannulae. These small clots partially occlude the blood circuit, but do not totally obstruct blood flow through the circuit. These clot restrictions increase the flow resistance and, thus increase the magnitude of pressure in the circuit. Accordingly, the common assumption that the flow resistance and pressure are constants in a blood circuit is not necessarily always valid. Gravity is another source of pressure change in a blood circuit. Gravity affects the pressure in a blood circuit. The pressure in the circuit will change due to gravity if the patient moves such that the height changes with respect to the vertical position of the circuit entry and exit points on the patient's arm relative to the pressure sensors. The pressure sensors in the blood circuit can detect a change that is indicative of a change in the patient's position, rather than a clot in the circuit. For example, if the patient sits up, pressure sensors will detect pressure changes of the blood in the circuit.

If the gravity induced pressure changes are sufficiently large, prior pump controllers tended to activate an alarm and shut down the blood pump. An operator must respond to the alarm, analyze the situation and remedy the malfunction or change the alarm limits if the flow path conditions have changed. In more advanced systems the operator sets the alarm window size (for example, plus or minus 50 mmHg about a mean point), and the machine will automatically adjust the mean point in proportion to the flow setting change. If the measured pressure exceeds the pressure range set by the window, the blood flow is automatically stopped. However, existing systems do not adjust the pump speed in response to changes in flow resistance, but rather, shut down if the resistance (and hence fluid pressure) become excessive.

### SUMMARY OF THE INVENTION

The present invention comprises blood measurement methods and apparatuses that include a blood flow controller that controls blood flow through an extracorporeal blood circuit. The controller regulates the flow rate through the circuit such that: (a) blood is withdrawn from a central or peripheral vein in the patient (peripheral veins are usually small, collapsible tubes) at a blood flow rate sustainable by the vein and which avoids collapsing the vein, and (b) blood pressure changes in the circuit are compensated for by adjusting pump speed and hence the flow rate through the circuit. The controller sets a flow rate of blood through the circuit based on both a maximum flow rate limit and a variable limit of pressure vs. flow rate. These limits can be preprogrammed in the controller, selected by an operator, or a combination thereof.

A novel blood measurement system according to the present invention enables rapid and safe recovery from occlusions in a withdrawal vein without participation of an operator, loss of circuits to clotting, or annoying alarms. The present invention provides a technique of compensating for and remedying temporary vein collapse when it occurs during blood withdrawal associated with measurement of blood characteristics. Not all episodes of a vein collapse require intervention from a doctor or nurse, or require that blood withdrawal cease for an extended period. For example, vein collapse can temporarily occur when the patient moves or a venous spasm causes the vein to collapse in a manner that is too rapid to anticipate and temporary. There has been a long-felt need for a control system for an extracorporeal circuit that can automatically recover from temporary occlusions. The present invention provides a system that can temporarily stop blood withdrawal when vein collapse occurs and, in some circumstances, infuse blood into the collapsed vein to reopen the collapsed vein.

Peripheral vein access presents unique problems that make it difficult for a blood withdrawal controller to maintain constant flow and not to create hazards for the patient. Using the present controller, for example, a patient can stand up during a measurement and thereby increase the static pressure height on the infusion side resulting in a false occlusion. The controller adjusts the blood flow rate through the extracorporeal circuit to accommodate for pressure changes. As the patient rises each centimeter (cm), the measured pressure in the extracorporeal circuit can increase by 0.73 mm Hg (milliliter of mercury). A change in height of 30 cm (approximately 1 ft) will result in a pressure change of 21 mm Hg. In addition, the patient can bend his/her arm during treatment and, thereby, reduce the blood flow to the withdrawal vein. As the flow through the withdrawal catheter decreases, the controller reduces pump speed to reduce the withdrawal pressure level. Moreover, the blood infusion operation of the blood circulation circuit can involve similar pressure variances. These infusion operation pressure changes can also be monitored by the controller which can adjust the pump to accommodate such changes.

The controller can be incorporated into a blood withdrawal and infusion pressure control system which optimizes blood flow at or below a preset rate in accordance with a controller algorithm that is determined for each particular make or model of an extraction and infusion extracorporeal blood system. The controller is further a blood flow control system that uses real time pressure as a feedback signal that is applied to control the withdrawal and infusion pressures within flow rate and pressure limits that are determined in real time as a function of the flow withdrawn from peripheral vein access.

The controller can govern the pump speed based on control algorithms and in response to pressure signals from pressure sensors that detect pressures in the blood flow at various locations in the extracorporeal circuit. One example of a control algorithm is a linear relationship between a minimum withdrawal pressure and withdrawal blood flow. Another example control algorithm is a maximum withdrawal flow rate. A control algorithm can be specified for the infusion pressure of the blood returned to the patient. In operation, the controller seeks a desired (e.g., maximum) blood flow rate that satisfies the control algorithms by monitoring the blood pressure during a withdrawal operation (and optionally during an infusion operation) of the blood circuit, and by controlling the flow rate of a variable pump. The controller can use the highest anticipated resistance for the circuit and therefore need not adjust flow until this resistance has been exceeded. If the maximum flow rate results in a pressure level outside of the pressure limit for the existing flow rate, the controller responds by reducing the flow rate, such as by reducing the speed of a roller pump, until the pressure in the circuit is no greater than the minimum (or maximum for infusion) variable pressure limit. The controller can automatically adjust the pump speed to regulate the flow rate and the pressure in the circuit. In this manner, the controller maintains the blood pressure in the circuit within both the flow rate limit and the variable pressure limits that have been preprogrammed or entered in the controller.

In normal operation, the controller causes the pump to drive the blood through the extracorporeal circuit at a set flow rate (e.g., a maximum rate). In addition, the controller monitors the pressure to ensure that it conforms to the programmed variable pressure vs. flow limit. Each pressure vs. flow limit prescribes a minimum (or maximum) pressure in a defined portion of the system as a function of blood flow rate. If the blood pressure falls or rises beyond the pressure limit for a current flow rate, the controller adjusts the blood flow by adjusting the pump speed. With a reduced blood flow, the pressure should rise during a withdrawal operation (or fall in an infusion operation). The controller can continue to adjust the pump speed until the pressure conforms to the pressure limit for the then-current flow rate.

When the pressure of the adjusted blood flow, e.g. a reduced flow, is no less than (or no greater than) the pressure limit for that new flow rate (as determined by the variable pressure vs. flow condition), the controller maintains the pump speed and operation of the blood circuit at a constant rate. The controller can gradually advance the flow rate in response to an improved access condition, provided that the circuit remains in compliance with the maximum rate and the pressure vs. flow limit.

The controller has several advantages over the prior art including (without limitation): (1) the controller adjusts the pump speed to regulate the blood flow rate and maintain the blood pressure within prescribed limits, without requiring the attention of or adjustment by an operator; (2) the controller adjusts blood flow in accordance with an occlusion pressure limit that varies with flow rate; and (3) the controller adaptively responds to partial occlusions in the withdrawal blood flow. In addition, the controller can implement other safety features, such as detecting the occurrence of total unrecoverable occlusions in the circuit and disconnections of the circuit, which can cause the controller to interpret that blood loss is occurring through the extracorporeal circuit to the external environment and stop the pump.

The controller can also compensate for variances in flow restrictions using control algorithms that apply pressure targets such as a withdrawal occlusion pressure target and an infusion occlusion pressure target. By compensating for control targets, the controller can monitor and discretely or simultaneously adjust for flow restrictions, e.g., partial occlusions that occur in various lines of the blood circuit. For example, if an occlusion occurs in a withdrawal vein, the pressure drop in a withdrawal operation can be sensed by the controller which in turn can reduce the flow rate in accordance with an adjustable withdrawal pressure limit. The pump continues at a reduced speed, provided that the variable pressure vs. flow limit is satisfied and the boundary limits of the pressure vs. flow relationship are not exceeded. The controller might be required to stop the pump entirely, if the blood does not flow sufficiently (with acceptable pressure) at any pump speed. In such a condition, the controller will slow the pump to a stop as the withdrawal pressure target decreases. When the flow drops below a preprogrammed limit for a preprogrammed time, the pump can also be stopped by the flow controller and a user alarm will be activated.

In the event of a withdrawal pressure occlusion that terminates flow, the flow controller can temporarily reverse the flow of the pump in an attempt to remove the withdrawal occlusion. The occlusion algorithms are still active during this maneuver, and can also terminate flow if the occlusion cannot be removed. The volume displacement of the pump can be limited to a specific number of revolutions to ensure that the pump does not infuse air into the patient.

With respect to an infusion pressure target, if a partial occlusion occurs in an infusion vein, the pressure controller detects a pressure rise in the return line and reduces the infusion rate. The controller will continue to reduce the pump speed and reduce the pressure in the infusion line. If the occlusion is total, the controller will quickly reduce the pump speed to a stop and avoid excessive pressure being applied to the infusion vein. The controller can also activate an alarm whenever the pump speed is stopped (or reduced to some lower speed level).

An application of the controller is in an extracorporeal blood system that includes a blood measurement device in a blood circuit. The blood measurement device can be, for example, an optical or electrochemical glucose measurement device. The blood access system can allow for reliable, automatic blood sampling and reinfusion, enabling frequent blood measurements with very little blood loss or no blood loss.

The controller system can provide a mechanism for maintaining patient safety while preventing false alarms when blood pressure measurements are made with or without the use of a blood pressure cuff. The controller system can provide a method for determining the development of a partial occlusion as well as the ability to identify what portion of the blood circuit is experiencing changes in flow dynamics. The controller system can provide a method for minimizing the time associated with blood withdrawal by operating in a constant pressure mode.

Advantages and novel features will become apparent to those skilled in the art upon examination of the following description or can be learned by practice of the invention. The advantages of the invention can be realized and attained by means of the instrumentalities and combinations particularly pointed out in the appended claims.

### BRIEF DESCRIPTION OF THE DRAWINGS

FIG. 1 illustrates the treatment of a patient with an ultrafiltration system using a controller in accordance with the present invention to monitor and control pressure and flow in an extracorporeal blood circuit.

FIG. 2 illustrates the operation and fluid path of the blood circuit shown in FIG. 1.

FIG. 3 is a chart of the withdrawal occlusion and disconnect limits applied by the controller.

FIG. 4 is a flow chart of an algorithm to implement the occlusion and disconnect limits shown in FIGS. 3 and 6, and showing how the blood withdrawal and infusion occlusion and disconnect pressures are calculated as a function of measured blood flow.

FIG. 5 is a flow chart of an algorithm showing a blood withdrawal and infusion PIFF pressure control algorithm to be implemented by the controller.

FIG. 6 is a chart of infusion occlusion and disconnect limits for the ultrafiltration system.

FIG. 7 is a component diagram of the controller (including controller CPU, monitoring CPU and motor CPU), and of the sensor inputs and actuator outputs that interact with the controller.

FIG. 8 is an illustration of the system response to the partial occlusion of the withdrawal vein in a patient.

FIG. 9 is an illustration of the system response to the complete occlusion and temporary collapse of the withdrawal vein in a patient.

FIG. 10 illustrates the measurement of a blood characteristic in a patient using a controller in accordance with the present invention to monitor and control pressure and flow in an extracorporeal blood circuit used for measurement of blood characteristics.

FIG. 11 illustrates the operation and fluid path of the blood circuit shown in FIG. 10.

### DETAILED DESCRIPTION OF THE INVENTION

A pump controller has been developed which can be incorporated in an extracorporeal blood circuit system. This system in an exemplary embodiment withdraws blood from a peripheral vein of a patient, processes the blood, e.g., passes the blood through a pump and filter, and returns the blood to the same or another peripheral vein. The pump controller monitors the blood pressure in the blood circuit and adjusts the speed of the pump (and hence the blood flow rate through the circuit) to comply with multiple limits on the pressure level and flow rates in the circuit. In addition, the controller promptly reacts to any changes in the pressure in the circuit.

The withdrawal and infusion of blood from a peripheral vein (or peripheral artery) in a mammalian patient (whether the patient is a human or other mammal) presents unique problems, which have been successfully addressed by the controller disclosed here. A peripheral vein in a human is a hollow tube, having approximately a 2 to 4 mm internal diameter. The wall of the vein is soft, flexible and not structurally self-supporting.

Blood pressure in the vein is required to keep the blood passage open and blood flowing through the vein. In a human vein, normal blood pressure in a vein is between 5 and 20 mmHg (millimeters of mercury). The blood flow through a peripheral vein generally ranges between 50 and 200 ml/min (milliliters per minute). Maintaining adequate pressure in a blood vessel from which blood is being withdrawn ensures that the vessel remains open to the flow of blood. The vein will collapse if the pressure drops excessively in a blood vessel, e.g., a vein. If the pressure in the vein becomes sub-atmospheric, the outside atmospheric pressure acting on the body will cause the vein to collapse.

An extracorporeal blood circuit draws blood from a peripheral vein (or artery) by applying a low pressure to a blood withdrawal tube attached to a catheter inserted into the vein. The pressure in the withdrawal tube is lower than the blood pressure in the vein. Due to this low pressure, some blood in the vein is drawn into the catheter and withdrawal tube. The lower pressure in the withdrawal tube and catheter is created by a pump in the blood circuit system that draws blood through the circuit and, in doing so, reduces the pressure in the withdrawal tube that is upstream of the pump. The reduced pressure in the withdrawal tube also reduces the pressure in the catheter and in the peripheral vein in which the catheter needle is inserted.

The reduced pressure in the vein near the catheter creates a potential risk of withdrawing blood from a peripheral vein too quickly and collapsing the vein. If the rate of blood flow into the withdrawal catheter is too great, the blood pressure in the vein will drop below that pressure required to keep the vein open and the vein will begin to collapse. As the vein collapses around the catheter, the blood flow into the catheter and the blood circuit is gradually reduced due to restrictions (“occlusions”) in the collapsing vein. As the blood flow into the blood circuit decreases, the pressure in the withdrawal line drops further because the pump (if it remains at a constant speed) is still attempting to pull blood through the circuit at a constant rate. Thus, the pump can accelerate the collapse of the withdrawal vein by exacerbating the pressure drop in the vein, unless the speed of the pump is reduced before the vein fully collapses.

The novel pressure controller disclosed here prevents complete vein collapse by reducing the blood withdrawal flow rate in response to a pressure drop in a withdrawal tube. If the vein collapses nevertheless intermittently, the controller facilitates recovery and continues the blood withdrawal. A pressure sensor in the withdrawal tube monitors the blood pressure in real time. If and when a pressure drop is detected which exceeds the specified allowed limit in the withdrawal line, the controller (which receives and processes the pressure sensor signal) slows the blood pump to reduce the flow rate of blood being withdrawn from the peripheral vein. By slowing the withdrawal flow, the pressure in the withdrawal line and peripheral vein near the catheter may return to a higher level. This pressure increase will hopefully be sufficient to prevent vein collapse, before it actually occurs and allow for a continued withdrawal blood flow (albeit at a reduced withdrawal flow). However, if the pressure in the withdrawal line does not sufficiently elevate and the vein continues to fully collapse, the controller will detect the continued low pressure in the withdrawal line and continue to reduce the pump flow until the pump stops.

An embodiment of the present invention is a pump controller with a blood measurement system that withdraws blood from peripheral blood vessels. The controller includes a microprocessor and memory for storing data and software control algorithms. The microprocessor receives input signals from pressure sensors regarding the blood pressure in the extracorporeal circuit, and from the pump regarding the pump speed. The microprocessor processes these input signals, applies the control algorithms and generates control signals that regulate the pump and hence the flow rate of blood through the circuit.

The controller may regulate blood withdrawn from a peripheral vein to a flow rate in a normal range of 0 to 150 ml/min (milliliters per minute). An operator may select a maximum withdrawal flow rate within this normal pressure range at which the blood measurement system is to operate. The controller will maintain the flow rate at or near the desired flow rate, provided that there is compliance with a pressure vs. flow rate limit control algorithm. The controller maintains the withdrawal blood flow rate at the selected flow rate, but automatically reduces the flow rate if the pressure in the system falls below a pressure limit for the actual flow rate. Thus, if there develops a partial flow restriction in the withdrawal vein or in the extracorporeal system, the controller will react by reducing the flow rate.

The controller optimizes blood flow at or below a preset maximum flow rate in accordance with one or more pressure vs. flow algorithms. These algorithms may be stored in memory of the controller which includes a processor, e.g., microprocessor; memory for data and program storage; input/output (I/O) devices for interacting with a human operator, for receiving feedback signals, e.g., pressure signals, from the blood circuit and possibly other systems, e.g., patient condition, and for issuing commands to control the pump speed; and data busses to allow the controller components to communicate with one another.

The control algorithms can include (without limitations): maximum flow settings for an individual patient treatment that is entered by the operator, a data listing of acceptable withdrawal/line pressures for each of a series of flow rates, and mathematical equations, e.g., linear, which correlates acceptable pressure to a flow rate. The algorithms may be determined for each particular make or model of an extraction and infusion extracorporeal blood system. In the present embodiment, the pressure vs. flow rate curves for occlusion and disconnect for the specified blood circuits are preprogrammed into the system.

Feedback signals are also used by the controller to confirm that the control algorithms are being satisfied. A real time pressure sensor signal from the withdrawal tube may be transmitted (via wire or wireless) to the controller. This pressure signal is applied by the controller as a feedback signal to compare the actual pressure with the pressure limits stored in memory of the controller for the current flow rate through the blood circuit. Based on this comparison, the controller sends control commands to adjust the speed of the pump motor, which controls the withdrawal and infusion pressures in the blood circuit. Using the pressure feedback signal, the controller ensures that the flow rate in the circuit complies with the variable pressure limits. Moreover, the pressure is monitored in real time every 10 ms so that the controller may continually determine whether the flow rate/pressure is acceptable. This is achieved by looking at the average flow rate over a consecutive one second period, and if the flow is less than a preset rate, the pump is stopped.

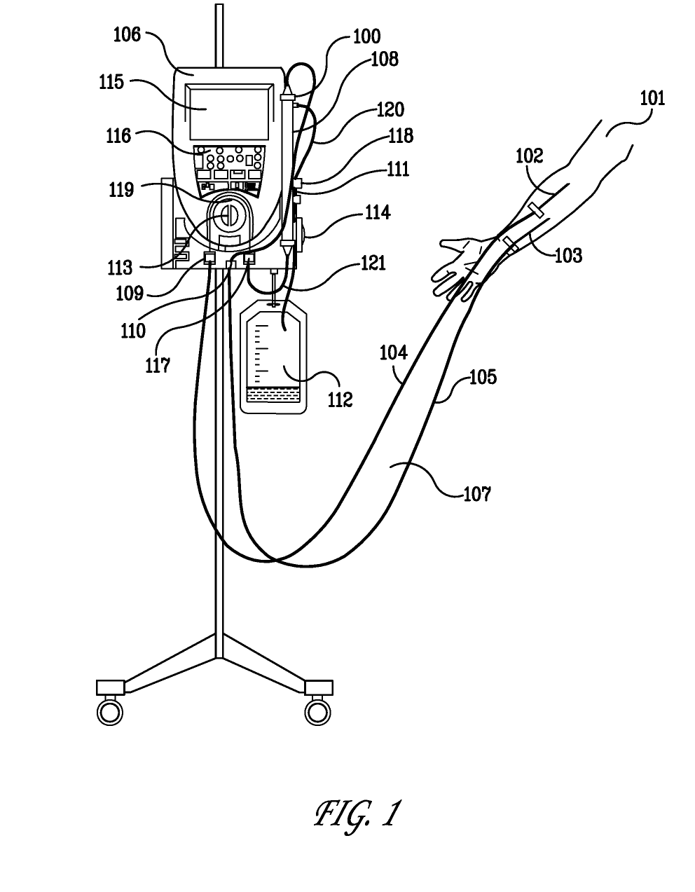

An exemplary apparatus described here is an ultrafiltration apparatus designed for the extraction of plasma water from human blood. To extract plasma water (ultrafiltrate), the apparatus includes a filter. The filter has a membrane that is permeable to water and small molecules, and impermeable to blood cells, proteins and other large solutes particles. FIG. 1 illustrates the treatment of a fluid overloaded patient with an ultrafiltration apparatus 100. The patient 101, such as a human or other mammal, can be treated while in bed or sitting in a chair and may be conscious or asleep. The apparatus can be attached to the patient in a doctor's office, an outpatient clinic, and may even be suitable for use at home (provided that adequate supervision of a doctor or other medically trained person is present). The patient need not be confined to an intensive care unit (ICU), does not require surgery to be attached to the ultrafiltration apparatus, and does not need specialized care or the continual presence of medical attendants.

To initiate ultrafiltration treatment, two standard 18G (gage) catheter needles, a withdrawal needle 102 and an infusion (return) needle 103, are introduced into suitable peripheral veins (on the same or different arms) for the withdrawal and return of the blood. This procedure of inserting needles is similar to that used for inserting catheter needles to withdraw blood or for intravenous (IV) therapy. The needles can be attached to withdrawal tubing 104 and return tubing 105, respectively. The tubing can be secured to skin with adhesive tape.

The ultrafiltration apparatus includes a blood pump console 106 and a blood circuit 107. The console includes two rotating roller pumps that move blood and ultrafiltrate fluids through the circuit, and the circuit is mounted on the console. The blood circuit includes a continuous blood passage between the withdrawal catheter 102 and the return catheter 103. The blood circuit includes a blood filter 108; pressure sensors 109 (in withdrawal tube), 110 (in return tube) and 111 (in filtrate output tube); an ultrafiltrate collection bag 112 and tubing lines to connect these components and form a continuous blood passage from the withdrawal to the infusion catheters an ultrafiltrate passage from the filter to the ultrafiltrate bag. The blood passage through the circuit is preferably continuous, smooth and free of stagnate blood pools and air/blood interfaces. These passages with continuous airless blood flow reduce the damping of pressure signals by the system and allows for a higher frequency response pressure controller, which allows the pressure controller to adjust the pump velocity more quickly to changes in pressure, thereby maintaining accurate pressure control without causing oscillation. The components of the circuit can be selected to provide smooth and continuous blood passages, such as a long, slender cylindrical filter chamber, and pressure sensors having cylindrical flow passage with electronic sensors embedded in a wall of the passage. The circuit can come in a sterile package and is intended that each circuit be used for a single treatment. A more detailed description of an exemplary blood circuit is included in commonly owned and co-pending (U.S. patent application Ser. No. 09/660,195, filed Sep. 12, 2000, which is incorporated by reference.

The circuit mounts on the blood and ultrafiltrate pumps 113 (for blood passage) and 114 (for filtrate output of filter). The circuit can be mounted, primed and prepared for operation within minutes by one operator. The operator of the blood ultrafiltration apparatus 100, e.g., a nurse or medical technician, sets the maximum rate at which fluid is to be removed from the blood of the patient. These settings are entered into the blood pump console 106 using the user interface, which may include a display 115 and control panel 116 with control keys for entering maximum flow rate and other controller settings. Information to assist the user in priming, setup and operation is displayed on the LCD (liquid crystal display) 115.

The ultrafiltrate is withdrawn by the ultrafiltrate pump 114 into a graduated collection bag 112. When the bag is full, ultrafiltration stops until the bag is emptied. The controller can determine when the bag is filled by determining the amount of filtrate entering the bag based on the volume displacement of the ultrafiltrate pump in the filtrate line and filtrate pump speed, or by receiving a signal indicative of the weight of the collection bag. As the blood is pumped through the circuit, an air detector 117 monitors for the presence of air in the blood circuit. A blood leak detector 118 in the ultrafiltrate output monitors for the presence of a ruptured filter. Signals from the air detector and/or blood leak detector can be transmitted to the controller, which in turn issues an alarm if a blood leak or air is detected in the ultrafiltrate or blood tubing passages of the extracorporeal circuit.

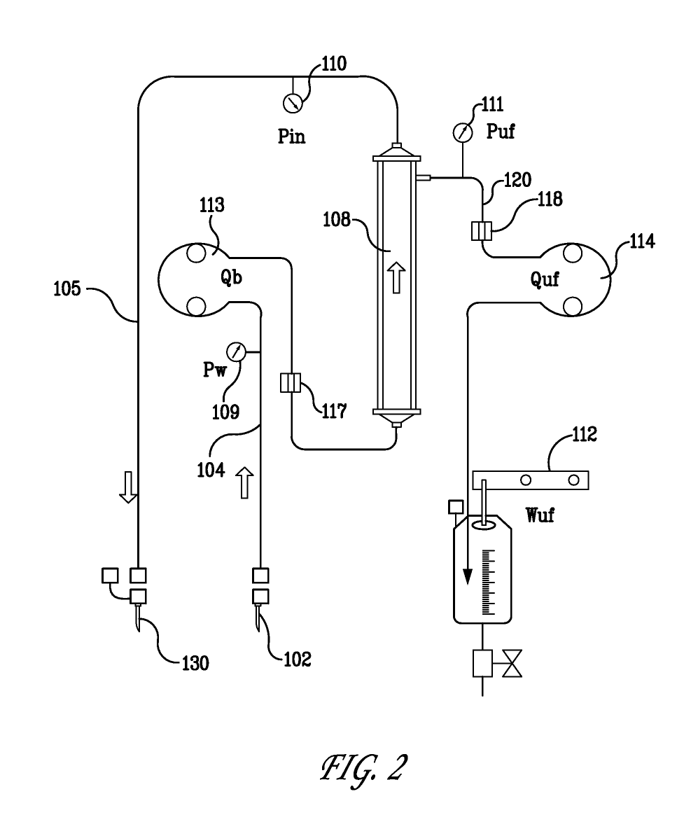

FIG. 2 illustrates the operation and fluid paths of blood and ultrafiltrate through the blood circuit 107. Blood is withdrawn from the patient through an 18 Gage or similar withdrawal needle 102. The withdrawal needle 102 is inserted into a suitable peripheral vein in the patient's arm. The blood flow from the peripheral vein into the withdrawal tubing 104 is dependent on the fluid pressure in that tubing which is controlled by a roller pump 113 on the console 106.

The length of withdrawal tubing between the withdrawal catheter and pump 113 can be approximately two meters. The withdrawal tubing and the other tubing in the blood circuit may be formed of medical PVC (polyvinyl chloride) of the kind typically used for IV (intravenous) lines which generally has an internal diameter (ID) of 3.2 mm. IV line tubing can form most of the blood passage through the blood circuit and have a generally constant ID throughout the passage.

The pressure sensors can also have a blood passage that is contiguous with the passages through the tubing and the ID of the passage in the sensors can be similar to the ID in the tubing. It is preferable that the entire blood passage through the blood circuit (from the withdrawal catheter to the return catheter) have substantially the same diameter (with the possible exception of the filter) so that the blood flow velocity is substantially uniform and constant through the circuit. A benefit of a blood circuit having a uniform ID and substantially continuous flow passages is that the blood tends to flow uniformly through the circuit, and does not form stagnant pools within the circuit where clotting may occur.

The roller blood pump 113 is rotated by a brushless DC motor housed within the console 106. The pump includes a rotating mechanism with orbiting rollers that are applied to a half-loop 119 in the blood passage tubing of the blood circuit. The orbital movement of the rollers applied to tubing forces blood to move through the circuit. This half-loop segment can have the same ID as does the other blood tubing portions of the blood circuit. The pump can displace approximately 1 ml (milliliter) of blood through the circuit for each full orbit of the rollers. If the orbital speed of the pump is 60 RPM (revolutions per minute), then the blood circuit can withdraw 60 ml/min of blood, filter the blood and return it to the patient. The speed of the blood pump 113 may be adjusted by the controller to be fully occlusive until a pressure limit of 15 psig (pounds per square inch above gravity) is reached. At pressures greater than 15 psig, the pump rollers relieve because the spring force occluding the tube will be exceeded and the pump flow rate will no longer be directly proportional to the motor velocity because the rollers will not be fully occlusive and will be relieving fluid. This safety feature ensures the pump is incapable of producing pressure that could rupture the filter.

The withdrawal pressure sensor 109 is a flow-through type sensor suitable for blood pressure measurements. It is preferable that the sensor have no bubble traps, separation diaphragms or other features included in the sensor that might cause stagnant blood flow and lead to inaccuracies in the pressure measurement. The withdrawal pressure sensor can be designed to measure negative (suction) pressure down to 400 mm Hg.

All pressure measurements in the fluid extraction system are referenced to both atmospheric and the static head pressure offsets. The static head pressure offsets arise because of the tubing placement and the pressure sensor height with respect to the patient connection. The withdrawal pressure signal is used by the microprocessor control system to maintain the blood flow from the vein and limit the pressure. Typically, a peripheral vein can continuously supply between 60-200 ml/min of blood. This assumption is supported by the clinical experience with plasma apheresis machines.

A pressure sensor 121 can be included in the circuit downstream of the pumps and upstream of the filter. Blood pressure in the post pump, pre-filter segment of the circuit is determined by the patient's venous pressure, the resistance to flow generated by the infusion catheter 103, resistance of hollow fibers in the filter assembly 108, and the flow resistance of the tubing in the circuit downstream of the blood pump 113. At blood flows of 40 to 60 ml/min, in this embodiment, the pump pressure may be generally in a range of 300 to 500 mm Hg depending on the blood flow, condition of the filter, blood viscosity and the conditions in the patient's vein.

The filter 108 is used to ultrafiltrate the blood and remove excess fluid from the blood. Whole blood enters the filter and passes through a bundle of hollow filter fibers in a filter canister. There can be approximately 700 to 900 hollow fibers in the bundle, and each fiber is a filter. In the filter canister, blood flows through an entrance channel to the bundle of fibers and enters the hollow passage of each fiber. Each individual fiber has approximately 0.2 mm internal diameter. The walls of the fibers are made of a porous material. The pores are permeable to water and small solutes, but are impermeable to red blood cells, proteins and other blood components that are larger than 50,000-60,000 Daltons. Blood flows through the fibers tangential to the surface of the fiber filter membrane. The shear rate resulting from the blood velocity is high enough such that the pores in the membrane are protected from fouling by particles, allowing the filtrate to permeate the fiber wall. Filtrate (ultrafiltrate) passes through the pores in the fiber membrane (when the ultrafiltrate pump is rotating), leaves the fiber bundle, and is collected in a filtrate space between the inner wall of the canister and outer walls of the fibers.

The membrane of the filter acts as a restrictor to ultrafiltrate flow. An ultrafiltrate pressure transducer (Puf) 111 is placed in the ultrafiltrate line upstream of the ultrafiltrate roller pump 114. The ultrafiltrate pump 114 is rotated at the prescribed fluid extraction rate which controls the ultrafiltrate flow from the filter. Before entering the ultrafiltrate pump, the ultrafiltrate passes through approximately 20 cm of plastic tubing 120, the ultrafiltrate pressure transducer (Puf) and the blood leak detector 118. The tubing is made from medical PVC of the kind used for IV lines and has internal diameter (ID) of 3.2 mm. The ultrafiltrate pump 114 is rotated by a brushless DC motor under microprocessor control. The pump tubing segment (compressed by the rollers) has the same ID as the rest of the ultrafiltrate circuit.

The system can move through the filtrate line approximately 1 ml of filtrate for each full rotation of the pump. A pump speed of 1.66 RPM corresponds to a filtrate flow of 1.66 ml/min, which corresponds to 100 ml/hr of fluid extraction. The ultrafiltrate pump 114 is adjusted at the factory to be fully occlusive until a pressure limit of 15 psig is reached. The rollers are mounted on compression springs and relieved when the force exerted by the fluid in the circuit exceeds the occlusive pressure of the pump rollers. The circuit can extract 100 to 500 ml/hr of ultrafiltrate for the clinical indication of fluid removal to relieve fluid overload.

After the blood passes through the ultrafiltrate filter 108, it is pumped through a two meter infusion return tube 105 to the infusion needle 103 where it is returned to the patient. The properties of the filter 108 and the infusion needle 103 are selected to assure the desired TMP (Traris Membrane Pressure) of 150 to 250 mm Hg at blood flows of 40-60 ml/min where blood has hematocrit of 35 to 45% and a temperature of 34.degree. C. to 37.degree. C. The TMP is the pressure drop across the membrane surface and can be calculated from the pressure difference between the average filter pressure on the blood side and the ultrafiltration pressure on the ultrafiltrate side of the membrane. Thus, TMP=((Inlet Filter Pressure+Outlet Filter Pressure)/2)−Ultrafiltrate Pressure.

The blood leak detector 118 detects the presence of a ruptured/leaking filter, or separation between the blood circuit and the ultrafiltrate circuit. In the presence of a leak, the ultrafiltrate fluid will no longer be clear and transparent because the blood cells normally rejected by the membrane will be allowed to pass. The blood leak detector detects a drop in the transmissibility of the ultrafiltrate line to infrared light and declares the presence of a blood leak.

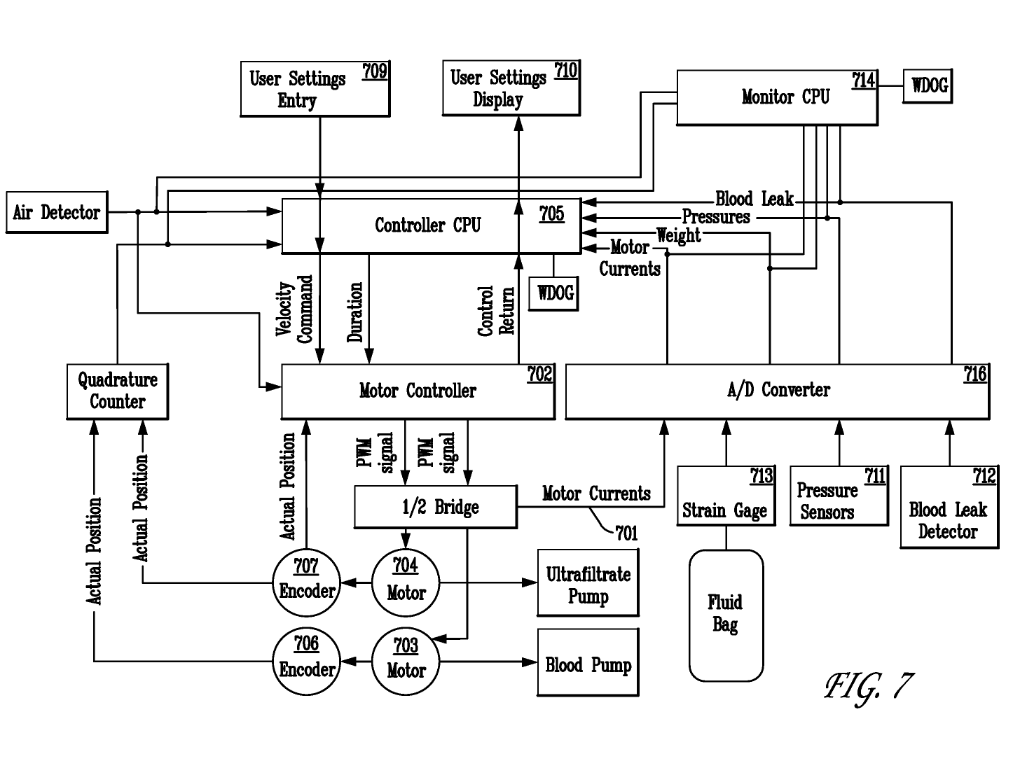

The pressure transducers Pw (withdrawal pressure sensor 109), Pin (infusion pressure sensor 110) and Puf (filtrate pressure sensor 111) produce pressure signals that indicate a relative pressure at each sensor location. Prior to filtration treatment, the sensors are set up by determining appropriate pressure offsets. These offsets are used to determine the static pressure in the blood circuit and ultrafiltrate circuit due to gravity. The offsets are determined with respect to atmospheric pressure when the blood circuit is filled with saline or blood, and the pumps are stopped. The offsets are measures of the static pressure generated by the fluid column in each section, e.g., withdrawal, return line and filtrate tube, of the circuit. During operation of the system, the offsets are subtracted from the raw pressure signals generated by the sensors as blood flows through the circuit. Subtracting the offsets from the raw pressure signals reduces the sensitivity of the system to gravity and facilitates the accurate measurement of the pressure drops in the circuit due to circuit resistance in the presence of blood and ultrafiltrate flow. Absent these offsets, a false disconnect or occlusion alarm could be issued by the monitor CPU (714 in FIG. 7) because, for example, a static 30 cm column of saline/blood will produce a 22 mm Hg pressure offset.

The pressure offset “Poffset” for a particular sensor is a function of the fluid density “rho”, the height of the tube “h” and the earth's gravitational constant “g”:

Poffset=rho*g*h

where “rho” and “g” are constants and, thus, pressure offsets are a function of the sensor position. The pressure offsets are not experienced by the patient. Proof of this is when a 3.2 mm ID tube filled with water with its top end occluded (pipette) does not allow the water to flow out. This means that the pressure at the bottom of the tube is at 0 mm Hg gage. In order to normalize the offset pressures, the offsets are measured at the start of operation when the circuit is fully primed and before the blood pump or ultrafiltrate pump are actuated. The measured offsets are subtracted from all subsequent pressure measurements. Therefore, the withdrawal pressure “Pw”, the infusion pressure “Pin” and the ultrafiltrate pressure “Puf” can be calculated as follows:

Pw=PwGage−PwOffset

Pin=PinGage−PinOffset

Puf=PufGage−PufOffset

PwOffset, PinOffset and PufOffset are measured when the circuit is primed with fluid, and the blood and ultrafiltrate pumps are stopped. PwGage, PinGage and PufGage are measured in real time and are the raw, unadjusted gage pressure readings from the pressure transducers. To increase accuracy and to minimize errors due to noise, the offsets are checked for stability and have to be stable within 2 mm Hg for 1 second before an offset reading is accepted. The offset is averaged over 1 second to further reduce sensitivity to noise.

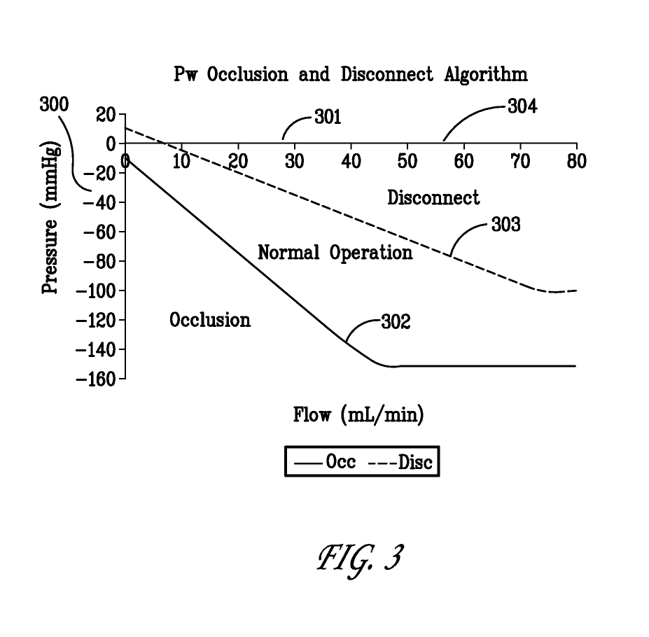

FIG. 3 is a chart of pressure limits 300 in the blood circuit versus the blood flow rate 301 in the circuit. The chart shows graphically exemplary control algorithms for controlling pressure in the withdrawal line as a function of the actual blood flow. The blood flow rate is known, and calculated from the known pump speed. An occlusion control function 302 (PwOcc-Occlusion) provides a variable pressure limit vs. flow rate (sloped portion of PwOcc-Occlusion) for controlling the minimum pressure limit in the withdrawal line as a function of flow rate.

The maximum negative pressure (i.e., lowest suction level) in the withdrawal line is limited by an algorithm 303 (disconnect-PwDisc) which is used to sense when a disconnect occurs in the withdrawal line. The withdrawal line has a suction pressure (sub-atmospheric) pressure to draw blood from the peripheral artery. This suction pressure is shown as a negative pressure in mmHg in FIG. 3. If the actual suction pressure rises above a limit (PwDisc), then the controller may signal that a disconnect has occurred, especially if air is also detected in the blood circuit. The suction pressure in the withdrawal line is controlled to be between the occlusion and disconnect pressure limits 302, 303.

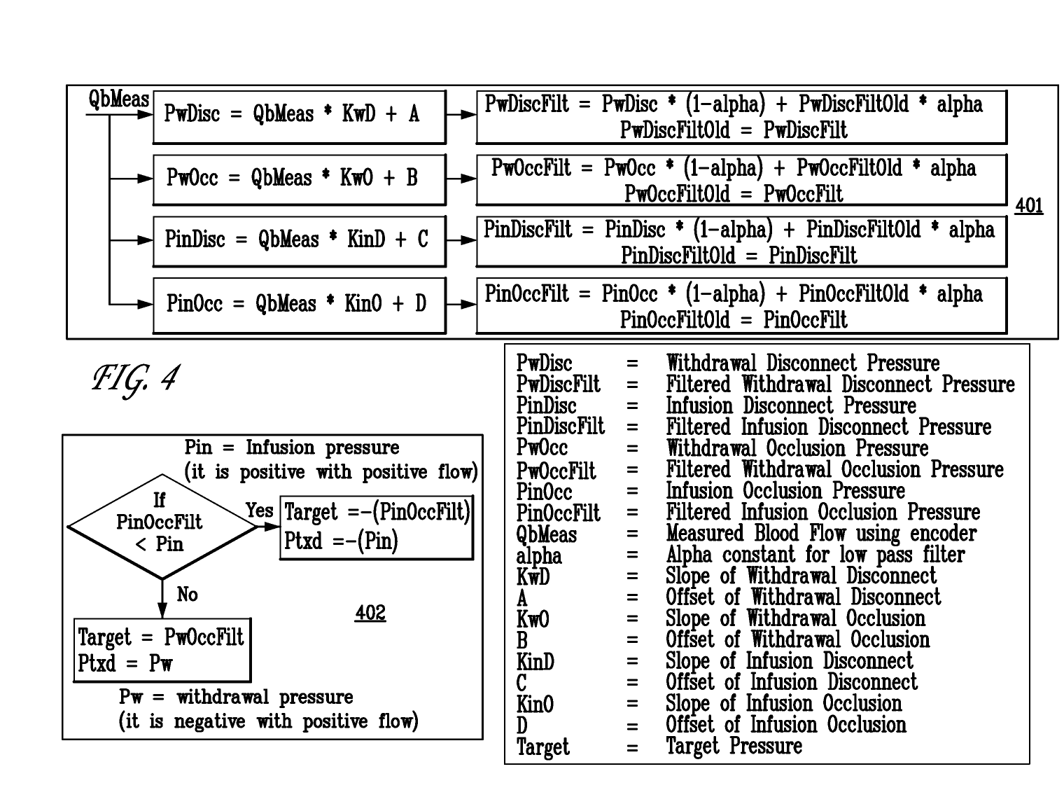

The maximum withdrawal resistance (PwOcc; see the slope of line 302) for a given flow rate is described by the occlusion algorithm curve 302. This allowable occlusion pressure, PwOcc (401 in FIG. 4), increases as blood flow increases. This increase may be represented by a linear slope of flow rate vs. pressure that continues until a maximum flow rate 304 is reached. The occlusion algorithm curve is based on theoretical and empirical data with a blood Hct of 35% (maximum Hct expected in clinical operation), and the maximum expected resistance of the withdrawal needle and withdrawal blood circuit tube expected during normal operation when measured at Pw.

The withdrawal pressure sensor signal (Pw) is also applied to determine whether a disconnection has occurred in the withdrawal blood circuit between the withdrawal tubing 104 from the needle 102 or between the needle and the patient's arm, or a rupture in the withdrawal tubing. The control algorithm for detecting a disconnection is represented by PwDisc curve 303. This curve 303 represents the minimum resistance of the 18 Gage needle and withdrawal tubing, with a blood Hct of 25% (minimum Hct expected in clinical operation), at a temperature of 37.degree. C. The data to generate this curve 303 may be obtained in vitro and later incorporated in the controller software.

During the device operation the measured withdrawal pressure (Pw) is evaluated in real time, for example, every 10 milliseconds, by the controller. Measured Pw is compared to the point on the curve 303 that corresponds to the current blood flow rate. A disconnection is detected when the pressure Pw at a given blood flow is greater than the pressure described by curve 303, and if air is detected in the blood circuit. If the withdrawal line becomes disconnected, the blood pump 113 will entrain air into the tubing due to the suction caused by the withdrawal pressure (Pw) when the blood pump is withdrawing blood. The pressure measured by the withdrawal pressure transducer Pw will increase (become less negative) in the presence of a disconnection because the resistance of the withdrawal line will decrease.

FIG. 4 is a flow chart showing in mathematical terms the control algorithms shown in FIG. 3. The allowable occlusion pressure (PwOcc) 401 is determined as a function of blood flow (QbMeas). The blood flow (QbMeas) may be determined by the controller, e.g., controller CPU, based on the rotational speed of the blood pump and the known volume of blood that is pumped with each rotation of that pump, as is shown in the equation below:

PwOcc=QbMeas*KwO+B

where QbMeas is the measured blood flow, KwO is the withdrawal occlusion control algorithm 302, e.g., a linear slope of flow vs. pressure, and B is a pressure offset applied to the withdrawal occlusion, which offset is described below.

The expression for PwOcc is a linear equation to describe. PwOcc may also be implemented as a look up table where a known QbMeas is entered to obtain a value for PwOcc. In addition, the expression for PwOcc may be a second order polynomial in the presence of turbulent flow. The expression for PwOcc to be chosen in a particular implementation will be based upon the characteristics of the tube and the presence of laminar or turbulent flow.

The PwOcc signal can be filtered with a 0.2 Hz low pass filter to avoid false occlusion alarms, as indicated in the following equations.

PcOccFilt=PwOcc*(1−alpha)+PwOccFiltOld*alpha

where alpha=exp(−t/Tau)
where t=discrete real time sample interval in second and the time constant Tau=1/(2*PI*Fc)
where PI is approximately 3.14159 and Fc is equal to the cutoff frequency of the first order low pass filter in Hz
thus, for a 0.2Hx filter, Tau=0.7957 therefore alpha=0.9875
where PwOccFilt is the current calculated occlusion pressure limit for the actual flow rate, after being filtered. PwOccFiltOld is the previous calculated occlusion pressure, and “alpha” is a constant of the low pass filter. Thus, PwOccFiltOld=PwOccFilt, for each successive determination of PwOccfilt.

Similar determinations are made for the calculated pressure limits for the filtered withdrawal disconnect limit (PwDiscFilt), filtered infusion disconnect limit (PinDiscFilt) and filtered infusion occlusion limit (PinOccFilt).

The PwDisc curve 303, shown in FIG. 3 is described in equation form below and shown in 401 of FIG. 4. The withdrawal disconnection pressure, PwDisc is calculated as a function (KwD) of blood flow, QbMeas which is measured blood flow calculated from the encoder pump speed signal.

PwDisc=QbMease*KwD+A

where A is a pressure constant offset, and KwD represents the slope of the PwDisc curve 303. In addition, the PwDisc (withdrawal pressure limit for disconnect) is filtered with a 0.2 Hz low pass filter to avoid false disconnect alarms, reference 401 in FIG. 4.

PwDisc is a linear equation to describe. PwDisc may also be implemented as a look up table where a known QbMeas is entered to obtain a value for QbMeas. In addition, the expression for PwDisc may be a second order polynomial in the presence of turbulent flow. The expression for PwDisc to be chosen in a particular implementation will be based upon the characteristics of the tube and the presence of laminar or turbulent flow.

PwDiscFilt=PwDisc*(1−alpha)+PwDiscFiltOld*alpha

PwDiscFiltOld=PwDiscFilt

Where alpha is a function of the filter.

The air detector 117 detects the presence of air when entrained. If the withdrawal pressure (Pw) exceeds (is less negative than) the disconnect pressure (PwDisc) 303 and air is detected in the blood circuit by the air detector, then the controller declares a withdrawal disconnection, and the blood pump and the ultrafiltrate pump are immediately stopped. This logic function is expressed as: If (Pw>PwDiscFilt AND AirDetected=TRUE) then Declare a withdrawal disconnect.

The above logic function is a reliable detection of a withdrawal line disconnection, while avoiding false alarms due to blood pressure measurements with blood pressure cuffs. For example, a false alarm could be generated when blood pressure cuffs are pressurized which causes an increased venous pressure and in turn lower withdrawal pressure. The lower withdrawal pressure caused by a blood pressure cuff might be interpreted by the controller as a disconnection resulting in false alarms, except for the logic requirement of air being detected.

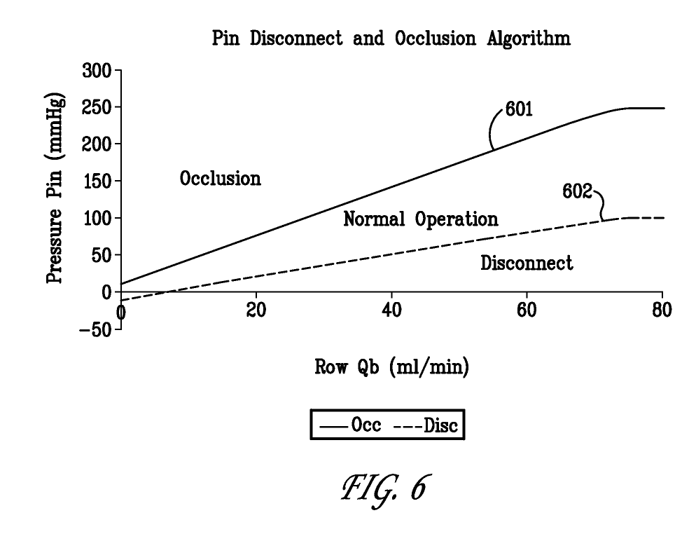

The occlusion and disconnect pressure limits for the return (infusion) line are graphically shown in FIG. 6. These calculations are made in a similar manner as described above for determining PwOccFilt. The infusion-occlusion pressure limit (PinOcc) 401 is calculated as a function of blood flow (QbMeas) where QbMeas is actual blood flow calculated from the pump speed feedback signal.

PinOcc=QbMeas*KwO+B, where KwO is the factor for converting (see FIG. 6, Occlusion line 601) the actual blood flow rate to a pressure limit. The expression for PinOcc is a linear equation to describe. PinOcc may also be implemented as a look up table where a known QbMeas is entered to obtain a value for PinOcc. In addition, the expression for PinOcc may be a second order polynomial in the presence of turbulent flow. The expression for PinOcc to be chosen in a particular implementation will be based upon the characteristics of the tube and the presence of laminar or turbulent flow. PinOcc is filtered with a 0.2 Hz low pass filter to avboid false disconnect alarms

PinOccFilt=PinOcc*(1−alpha)+PinOccfiltOld*alpha

PinOccFiltOld=PinOccFilt

FIG. 4 also shows the interaction of the control algorithms for withdrawal occlusion (PwOccFilt) and the infusion occlusion (PinOccFilt). The control theory for having two control algorithms applicable to determining the proper flow rate is that only one of the control algorithms will be applied to determine a target flow rate at any one time. To select which algorithm to use, the controller performs a logical “If-Then operation” 402 that determines whether the target is to be the withdrawal occlusion or infusion occlusion algorithms. The criteria for the If-Then operation is whether the infusion line is occluded or not. If the infusion line is occluded, Pin is greater than PinOccFilt; therefore, the Target is set to PinOccFilt.

In particular, the infusion occlusion algorithm (PinOccFilt) is the target (Target) and infusion pressure (Pin) is applied as a feedback signal (Ptxd), only when the infusion pressure (Pin) exceeds the occlusion limit for infusion pressure (PinOccFilt). Otherwise, the Target is the occlusion withdrawal pressure limit (PwOccFilt) and the feedback signal is the withdrawal pressure (Pw).

The If-Then (402) algorithm is set forth below in a logic statement (see also the flow chart 402):

### If (PinOccFilt<Pin)

### {Then Target=−(PinOccFilt), and Ptxd=−(Pin)}

### {Else Target=PwOccFilt and Ptxd=Pw}

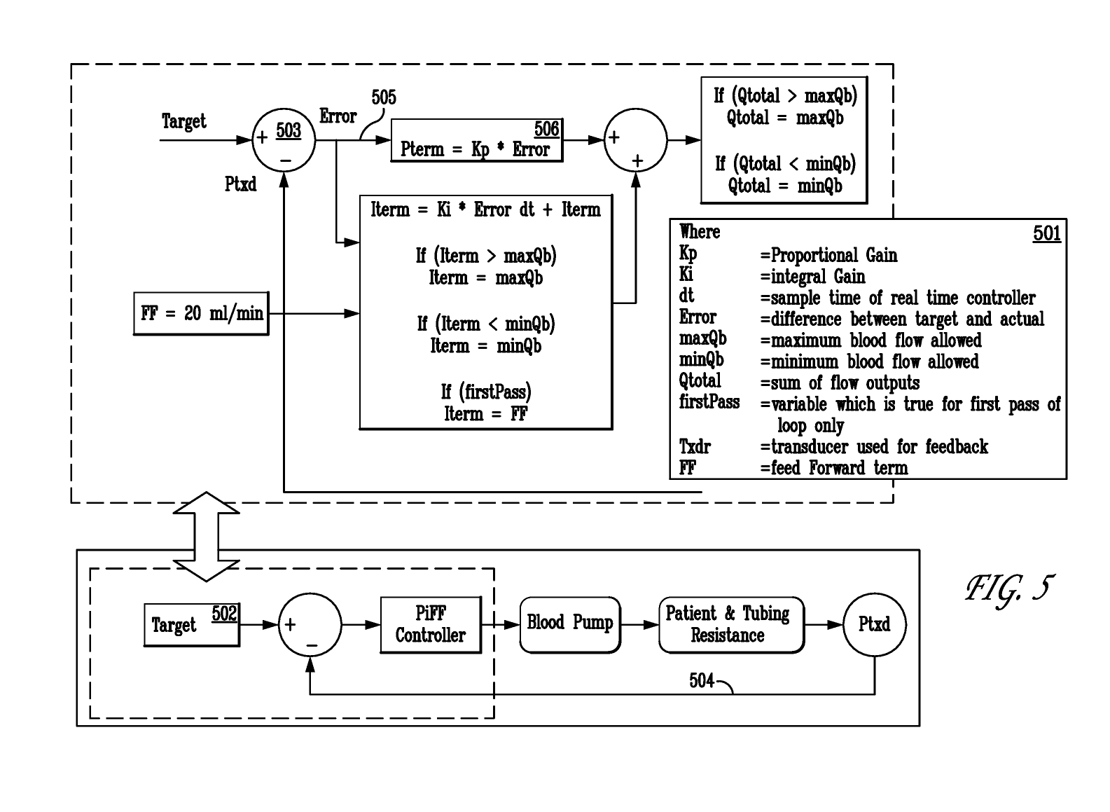

A pressure controller (see FIG. 5 description) can be used to control the Ptxd measurement to the Target pressure. The Target pressure will be either the PinOccFilt or PwOccFilt limit based upon the IF statement described above.

FIG. 5 includes a functional diagram of a PIFF (Proportional Integral Feed Forward) pressure controller 501 for the ultrafiltration apparatus 100, and shows how the PIFF operates to control pressure and flow of blood through the circuit. Controllers of the PIFF type are well known in the field of controls engineering. The PIFF pressure controller 501 controls the withdrawal pressure to the prescribed target pressure 502, which is the filtered withdrawal occlusion pressure limit (PwOccFilt), by adjusting the blood pump flow rate. The PIFF may alternatively use as a target the limit for infusion pressure (PinOccFilt). The target pressure 502 limit is compared 503 to a corresponding actual pressure 504, which is withdrawal pressure (Pw) if the target is PwOccFilt and is infusion pressure (Pin) if the target is PinOccFilt. The actual pressure is applied as a feedback signal (Ptxd) in the PIFF. The logical compare operation 503 generates a difference signal (Error) 505 that is processed by the PIFF.

The PIFF determines the appropriate total flow rate (Qtotal) based on the difference signal 505, the current flow rate, the current rate of increase or decrease of the flow rate, and the flow rate limit. The PIFF evaluates the difference between the target pressure limit and actual pressure (feedback) with a proportional gain (Kp), an integral gain (Ki) and a feed forward term (FF). The proportional gain (Kp) represents the gain applied to current value of the error signal 505 to generate a proportional term (Pterm) 506, which is one component of the sum of the current desired flow (Qtotal). The integral gain (Ki) is the other component of Qtotal, and is a gain applied to the rate at which the error signal varies with time (error dt). The product of the integral gain and the error dt (Iterm) is summed with the previous value of Iterm to generate a current item value. The current Iterm value and Pterm value are summed, checked to ensure that the sum is within flow limits, and applied as the current desired total flow rate (Qtotal). This desired flow rate (Qtotal) is then applied to control the blood pump speed, and, in turn, the actual flow rate through the blood circuit.

The gain of the PIFF pressure controller Kp and Ki have been chosen to ensure stability when controlling with both withdrawal and infusion pressures. The same PIFF controller is used for limiting withdrawal and infusion pressures. None of the controller terms are reset when the targets and feedback transducers are switched. This ensures that there are no discontinuities in blood flow and that transitions between control inputs are smooth and free from oscillation. Thus, when the PIFF pressure controller switches from controlling on withdrawal pressure top infusion pressure the blood pump does not stop, it continues at a velocity dictated by the pressure control algorithm.

The proportional and integral gains (Kp and Ki) of the pressure controller are selected to ensure stability. Kp and Ki were chosen to ensure that pressure overshoots are less than 30 mmHg, and that the pressure waveform when viewed on a data acquisition system was smooth and free of noise. In general Kp may be increased until the noise level on the signal being controlled exceeds the desired level. Kp is then reduced by 30%. Ki is chosen to ensure the steady state error is eliminated and that overshoot is minimized. Both the integral term and the total flow output of the PIFF controller are limited to a maximum of 60 ml/min, in this embodiment.

In addition, in this embodiment the flow limits for the integral term and total flow output may be increased linearly starting at a maximum rate of 20 ml/min (FF). When the PIFF controller is initially started, the integral term (Iterm) is set equal to the feed forward term (FF), which may be 20 ml/min. Thus, 40 seconds are required to increase the flow limit from an initial setting (20 ml/min) to the maximum value of 60 m/min. This 40 second flow increase period should be sufficient to allow the withdrawal vein to respond to increases in withdrawal flow rate. Limiting the rate of increase of the blood flow is needed because veins are reservoirs of blood and act as hydraulic capacitors. If a flow rate is increased too quickly, then a false high flow of blood can occur for short periods of time because flow may be supplied by the elastance of the vein (that determines compliance), and may not be true sustainable continuous flow much like an electrical capacitor will supply short surges in current.

This PIFF pressure controller controls pressure in real time, and will immediately reduce the pressure target if a reduction in flow occurs due to an occlusion. The target pressure is reduced in order to comply with the occlusion pressure limit, such as is shown in FIG. 3. Reducing the pressure target in the presence of an occlusion will lead to a further reduction in flow, which will result in a further reduction in the target pressure. This process limits the magnitude and duration of negative pressure excursions on the withdrawal side, and, therefore, exposure of the patient's vein to trauma. It also gives the withdrawal (or infusion) vein time to recover, and the patient's vein time to reestablish flow without declaring an occlusion.

When a withdrawal vein collapses, the blood pump will be stopped by the PIFF controller because the vein will have infinite resistance resulting in zero blood flow no matter to what pressure Pw is controlled. When the blood pump stops, the blood flow is reversed and blood is pumped into the withdrawal vein in an attempt to open that vein. When the blood pump is reversed, the withdrawal and infusion disconnect and occlusion algorithms are still active protecting the patient from exposure to high pressures and disconnects. When the blood pump flow is reversed the occlusion limits and disconnect limits are inverted by multiplying by negative 1. This allows the pump to reverse while still being controlled by maximum pressure limits. If (Blood Pump is reversing) {PwDiscFilt and PinDiscFilt and PwOccFilt and PwOccFilt are inverted}

Two (2) ml of blood can be infused into the withdrawal line and into the withdrawal peripheral vein by reversing the blood pump at 20 Ml/min to ensure that the vein is not collapsed. The blood pump is stopped for 2 seconds and withdrawal is reinitiated. The controller issues an alarm to request that the operator check vein access after three automatic attempts of reversing blood flow into the withdrawal line. The blood circuit has a total volume of approximately 60 ml. The blood pump is limited to reversing a total volume of five ml thereby minimizing the possibility of infusing the patient with air.

The PIFF applies a maximum withdrawal flow rate (maxQb) and a minimum withdrawal flow rate (minQb). These flow rate boundaries are applied as limits to both the integration term (Item) and the sum of the flow outputs (Qtotal). The maximum withdrawal rate is limited to, e.g., 60 ml/min, to avoid excessive withdrawal flows that might collapse the vein in certain patient populations. The minimum flow rate (minQb) is applied to the output flow to ensure that the pump does not retract at a flow rate higher than −60 ml/min. In addition, if the actual flow rate (Qb) drops below a predetermined rate for a certain period of time, e.g., 20 ml/min for 10 seconds, both the blood pump and ultrafiltrate pump are stopped.

The ultrafiltrate pump is stopped when the blood pump flow is less than 40 ml/min. If the ultrafiltrate pump is not stopped, blood can be condensed too much inside the fibers and the fibers will clot. A minimum shear rate of 1000 sec.sup.−1 in blood is desirable if fouling is to be avoided. This shear rate occurs at 40 ml/min in the 0.2 mm diameter filter fibers. The shear rate decreases as the flow rate decreases. Fouling may be due to a buildup of a protein layer on the membrane surface and results in an increase in trans-membrane resistance that can ultimately stop ultrafiltration flow if allowed to continue. By ensuring that no ultrafiltration flow occurs when a low blood shear rate is present, the likelihood of fouling is decreased.

When the system starts blood flow, the ultrafiltration pump is held in position and does not begin rotation until the measured and set blood flow are greater than 40 ml/min. If the set or measured blood flow drops below 40 ml/min, the ultrafiltrate pump is immediately halted. This prevents clogging and fouling. Once blood flow is re-established and is greater than 40 ml/min, the ultrafiltrate pump is restarted at the user defined ultrafiltration rate. When the blood pump is halted the ultrafiltrate pump is stopped first, followed by the blood pump ensuring that the filter does not become clogged because the ultrafiltrate pump was slower at stopping, resulting in ultrafiltrate being entrained while blood flow has ceased. This can be implemented with a 20 millisecond delay between halt commands.

FIG. 6 graphically shows the control algorithms for the blood infusion pressure. The patient can be exposed to excessively high pressures if an occlusion occurs in the infusion vein. Control algorithms are used for controlling the maximum allowable infusion pressure. These algorithms are similar in concept to those for controlling the maximum allowable withdrawal pressure. The maximum occlusion pressure algorithm 601 is a positive relationship between the flow rate (Qb) and infusion pressure (Pin) as measured by the pressure sensor in the return line 110. As shown in the algorithm curve 601, as the flow rate increases the acceptable infusion pressure similarly increases, up to a maximum limit.

The algorithm curve 601 provides the maximum infusion pressure, Pin, for given blood flow. The maximum allowable positive pressure PinOcc increases as blood flow Qb increases. This curve was generated from theoretical and empirical data with a blood Hct of 45% (maximum expected clinically), and is based on the maximum resistance of the infusion tubing 105 and the infusion needle 103. The curve can vary with different embodiments, depending on other data used to generate such a curve.

FIG. 7 illustrates the electrical architecture of the ultrafiltrate system 700 (100 in FIG. 1), showing the various signal inputs and actuator outputs to the controller. The user-operator inputs the desired ultrafiltrate extraction rate into the controller by pressing buttons on a membrane interface keypad 709 on the controller. These settings can include the maximum flow rate of blood through the system, maximum time for running the circuit to filter the blood, the maximum ultrafiltrate rate and the maximum ultrafiltrate volume. The settings input by the user are stored in a memory 715 (mem.), and read and displayed by the controller CPU 705 (central processing unit, e.g., microprocessor or micro-controller) on the display 710.

The controller CPU regulates the pump speeds by commanding a motor controller 702 to set the rotational speed of the blood pump 113 to a certain speed specified by the controller CPU. Similarly, the motor controller adjusts the speed of the ultrafiltrate pump 111 in response to commands from the controller CPU and to provide a particular filtrate flow velocity specified by the controller CPU. Feedback signals from the pressure transducer sensors 711 are converted from analog voltage levels to digital signals in an A/D converter 716. The digital pressure signals are provided to the controller CPU as feedback signals and compared to the intended pressure levels determined by the CPU. In addition, the digital pressure signals can be displayed by the monitor CPU 714.

The motor controller 702 controls the velocity, rotational speed of the blood and filtrate pump motors 703, 704. Encoders 707, 706 mounted to the rotational shaft of each of the motors as feedback provide quadrature signals, e.g., a pair of identical cyclical digital signals, but 90.degree. out-of-phase with one another. These signal pairs are fed to a quadrature counter within the motor controller 702 to give both direction and position. The direction is determined by the signal lead of the quadrature signals. The position of the motor is determined by the accumulation of pulse edges. Actual motor velocity is computed by the motor controller as the rate of change of position. The controller calculates a position trajectory that dictates where the motor must be at a given time and the difference between the actual position and the desired position is used as feedback for the motor controller. The motor controller then modulates the percentage of the on time of the PWM signal sent to the one-half 718 bridge circuit to minimize the error. A separate quadrature counter 717 is independently read by the Controller CPU to ensure that the Motor Controller is correctly controlling the velocity of the motor. This is achieved by differentiating the change in position of the motor over time.

The monitoring CPU 714 provides a safety check that independently monitors each of the critical signals, including signals indicative of blood leaks, pressures in blood circuit, weight of filtrate bag, motor currents, air in blood line detector and motor speed/position. The monitoring CPU has stored in its memory safety and alarm levels for various operating conditions of the ultrafiltrate system. By comparing these allowable preset levels to the real-time operating signals, the monitoring CPU can determine whether a safety alarm should be issued, and has the ability to independently stop both motors and reset the motor controller and controller CPU if necessary.

Peripheral vein access presents unique problems that make it difficult for a blood withdrawal controller to maintain constant flow and to not create hazards for the patient. For example, a patient might stand up during treatment and thereby increase the static pressure head height on the infusion side of the blood circuit. As the patient rises each centimeter (cm), the measured pressure in the extracorporeal circuit increases by 0.73 mm Hg. This static pressure rise (or fall) will be detected by pressure sensors in the withdrawal tube. The controller adjusts the blood flow rate through the extracorporeal circuit to accommodate for such pressure changes and ensures that the changes do not violate the pressure limits set in the controller.

In addition, the patient might bend their arm during treatment, thereby reducing the blood flow to the withdrawal vein. As the flow through the withdrawal catheter decreases, the controller reduces pump speed to reduce the withdrawal pressure level. Moreover, the blood infusion side of the blood circulation circuit might involve similar pressure variances. These infusion side pressure changes are also monitored by the controller which may adjust the pump flow rate to accommodate such changes.

In some cases, blood flow can be temporarily impeded by the collapse of the withdrawal vein caused by the patient motion. In other cases the withdrawal vein of the patient may not be sufficient to supply the maximum desired flow of 60 ml/min. The software algorithms enable the controller to adjust the withdrawal flow rate of blood to prevent or recover from the collapse of the vein and reestablish the blood flow based on the signal from the withdrawal pressure sensor.

A similar risk of disconnection exists when returning the patient's blood. The infusion needle or the infusion tube between the outlet of the infusion pressure transducer (Pin) and needle might become disconnected during operation. A similar disconnection algorithm (as described for the withdrawal side) is used for detecting the presence of disconnections on the infusion side. In this case an air detector is not used because nursing staff do not place pressure cuffs on the arm being infused because of risk of extravasation. Since the blood is being infused the pressures measured by the infusion pressure transducer Pin are positive. The magnitude of Pin will decrease in the presence of a disconnection due to a decrease in the resistance of the infusion line.

A disconnection is detected when the pressure Pin at a given blood flow is less than the pressure described by curve 602 (FIG. 6) for the same said blood flow. The minimum resistance of the 18 Gage needle and withdrawal tubing, with a blood Hct of 35%, at a temperature of 37.degree. C. are represented by the curve 602. The curve 602, shown in FIG. 6 is described in equation form in 401 (FIG. 4). The infusion disconnection pressure, PinDisc 401 is calculated as a function of blood flow, QbMeas where QbMeas, is actual blood flow calculated from the encoder velocity.

PinDisc=QbMeas*KinD+C

PinDisc is filtered with a 0.2 Hz low pass filter to avoid false disconnection alarms, reference 401 FIG. 4. The present embodiment uses a linear equation to describe PinDisc, but this equation could also be implemented as a look-up table or a second order polynomial in the presence of turbulent flow. The implementation chosen will be based upon the characteristics of the tube and the presence of laminar or turbulent flow.

PinDiscFilt=PinDisc*(1−alpha)+PinDiscFiltOld*alpha

PinDiscFiltOld=PinDiscFilt

If Pin is less than PinDiscFilt for 2 seconds consecutively, an infusion disconnect is declared and the blood pump and ultrafiltrate pump are immediately stopped.

### If (Pin>PinDiscFilt)

### {Then Increment Infusion Disconnect Timer}

{else Reset Infusion Disconnect Timer}
If (Reset Infusion Disconnect Timer=2 seconds)
{then Declare Infusion Disconnection}

The withdrawal and infusion occlusion detection algorithms use similar methods of detection. Only the specific coefficients describing the maximum and minimum allowable resistances are different.

The purpose of the withdrawal occlusion algorithm is to limit the pressure in the withdrawal vein from becoming negative. A negative pressure in the withdrawal vein will cause it to collapse. The venous pressure is normally 15 mm Hg and it will remain positive as long as the flow in the vein is greater than the flow extracted by the blood pump.

If the resistance of the withdrawal needle and blood circuit tube are known, the withdrawal flow may be controlled by targeting a specific withdrawal pressure as a function of desired flow and known resistance. For example, assume that the resistance of the withdrawal needle to blood flow is R and that R equals −1 mm Hg/ml/min. In order for 60 ml/min of blood to flow through the needle, a pressure drop of 60 mm Hg is required. The pressure may be either positive, pushing blood through the needle or negative, withdrawing blood through the needle. On the withdrawal side of the needle, if a pressure of −60 mm Hg is targeted a blood flow of 60 ml/min will result.

If the flow controller is designed to be based upon resistance, the pressure target required to give the desired flow rate Q would be R*Q. Thus, if a flow of 40 ml/min were required, a pressure of −40 mm Hg would be required as the pressure target. Since the system knows withdrawal flow based upon encoder velocity and is measuring withdrawal pressure, the system is able to measure the actual withdrawal resistance of the needle in real time.

If a maximum resistance limit is placed on the withdrawal needle of −1.1 mm Hg/ml/min, the pressure controller will stop withdrawing flow in the presence of an occlusion. Occlusion can be in the circuit or caused by the vein collapse. The resistance limit is implemented as a maximum pressure allowed for a given flow. Thus, for a resistance limit of −1.1 mm Hg, if the flow drops to 30 ml/min when the current withdrawal pressure is −60 mm Hg in the presence of an occlusion, the maximum pressure allowed is 30 ml/min*−1.1 mm Hg/ml/min=33 mm Hg. This means that the occlusion resistance is −60/30=−mm Hg/ml/min. If the occlusion persists when the withdrawal pressure drops to −33 mm Hg, the flow will be reduced to −16.5 ml/min. This will result in a new pressure target of −18.15 mm Hg and so on until the flow stops.

The actual pressure target to deliver the desired flow is difficult to ascertain in advance because of the myriad of variables which effect resistance, blood Hct, needle size within and length within the expected tolerance levels, etc. Instead, the pressure controller targets the maximum resistance allowed, and the flow is limited by the maximum flow output allowed by the pressure controller.

The control algorithm can ensure that the pressure at the withdrawal vein never falls below 0 mmHg where vein collapse could occur, or that the infusion pressure exceeds a value that could cause extravasation. If the critical pressure-flow curve is generated at the worst case conditions (highest blood viscosity), the controller will ensure that the pressure in the vein is always above the collapse level or below the extravasation level.

In the fluid path configuration of the blood circuit shown in FIG. 2, there is no pressure transducer at the blood pump outlet and entry to the filter. If a pressure transducer were present, then its signal could also be fed to the PIFF pressure controller using the same pressure limitation methods already described. A specific disconnect and occlusion algorithm could be defined to describe the maximum and minimum flow vs. pressure curves based upon the filter and infusion limb resistance. Alternatively, a limit on the current consumed by the motor can be used to detect the presence of an occlusion in the infusion and filter limb. High pressures at the filter inlet will not be detected by the infusion pressure transducer, (Pin), because it is downstream of this potential occlusion site. A disconnection at the inlet to the filter will be detected by the infusion disconnection algorithm, because if the filter becomes disconnected there will be no flow present in the infusion limb and this will be interpreted by the infusion disconnection algorithm as an infusion disconnection.

The blood pump uses a direct drive brushless DC motor. This design was chosen for long life and efficiency. Using this approach has the added benefit of being able to measure pump torque directly. With DC motors the current consumed is a function of motor velocity and torque. The current consumed by the motor may be measured directly with a series resistor, as indicated by 701, FIG. 7. This current is a function of the load torque and back EMF generated by the motor as a function of its speed and voltage constant. Thus:

Tmotor=Tpump+Ttube (equation 1)

Where Tmotor is the torque required to drive the motor, Tpump is the torque required to overcome the pressure in the tubing, T.sub.tube is the torque required to compress the tube.

Tmotor=(Imotor−(RPM*Kv)/Rmotor)*KT (equation 2)

Tmotor=(Imotor−IEMF)*KT

where Tmotor=Torque oz-in, RPM=revs per min of motor, Imotor is the current consumed by the motor, Kv is the voltage constant of the motor Volts/rpm, Rmotor=electrical resistance of motor in ohms and KT=the torque constant of the motor in oz-in/amp. KV and KT are constants defined by the motor manufacturer. The RPM of the motor can be calculated from the change in position of the motor encoder. Thus, by measuring the current consumed by the motor, the torque produced by the motor can be calculated if the speed and physical parameters of the motor are known.

The torque consumed by the motor is a function of withdrawal pressure, the blood pump outlet pressure and the tubing compressibility. The torque required to compress the blood circuit tubing is relatively constant and is independent of the blood flow rate. A good indication of the blood pump outlet pressure may be calculated as a function of the current consumed by the blood pump motor and may be used to indicate the presence of a severe occlusion.

The pump pressure Pp can be expressed as:

Tpump=Tmotor−Ttube (equation 4)

Pp=Tpump*K (equation 5)

### Where K=A/R

Pp=(Tmotor−Ttube)*K

where Pp is the blood pump outlet pressure, Tmotor is the total torque output by the motor, Ttube is the torque required to squeeze the blood circuit tube K and is a conversion constant from torque to pressure. K is calculated by dividing the cross-sectional area of the blood circuit tube internal diameter 3.2 mm by the radius R of the peristaltic blood pump.

Since K and Ttube are constants for the system and the blood flow has a range of 40 to 60 ml/min also making the back EMF current approximately constant. The motor current may be used directly without any manipulation to determine the presence of an occlusion as an alternative to calculating Pp. Thus, when the current limit of the blood pump exceeds, for example 3 amps, both the blood pump and the ultrafiltrate pump are stopped.

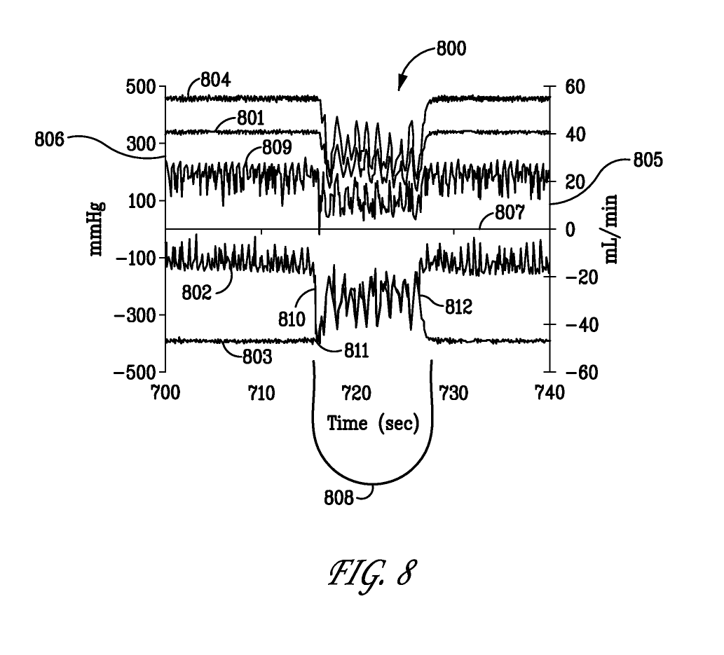

FIG. 8 illustrates the operation of a prototype apparatus constructed according to the current embodiment illustrated by FIGS. 1 to 7 under the conditions of a partial and temporary occlusion of the withdrawal vein. The data depicted in the graph 800 was collected in real time, every 0.1 second, during treatment of a patient. Blood was withdrawn from the left arm and infused into the right arm in different veins of the patient using similar 18 Gage needles. A short segment of data, i.e., 40 seconds long, is plotted in FIG. 8 for the following traces: blood flow in the extracorporeal circuit 804, infusion pressure occlusion limit 801 calculated by CPU 705, infusion pressure 809, calculated withdrawal pressure limit 803 and measured withdrawal pressure 802. Blood flow 804 is plotted on the secondary Y-axis 805 scaled in mL/min. All pressures and pressure limits are plotted on the primary Y-axis 806 scaled in mmHg. All traces are plotted in real time on the X-axis 807 scaled in seconds.

In the beginning, between time marks of 700 and 715 seconds, there is no obstruction in either infusion or withdrawal lines. Blood flow 804 is set by the control algorithm to the maximum flow limit of 55 mL/min. Infusion pressure 809 is approximately 150 to 200 mmHg and oscillates with the pulsations generated by the pump. Infusion occlusion limit 801 is calculated based on the measured blood flow of 55 mmHg and is equal to 340 mmHg. Similarly, the withdrawal pressure 802 oscillates between −100 and −150 mmHg safely above the dynamically calculated withdrawal occlusion limit 803 equal to approximately −390 mmHg.

At approximately 715 seconds, a sudden period of partial occlusion 808 occurred. The occlusion is partial because it did not totally stop the blood flow 804, but rather resulted in its significant reduction from 55 mL/min to between 25 and 44 mL/min. The most probable cause of this partial occlusion is that as the patient moved during blood withdrawal. The partial occlusion occurred at the intake opening of the blood withdrawal needle. Slower reduction in flow can also occur due to a slowing in the metabolic requirements of the patient because of a lack of physical activity. Squeezing a patient's arm occasionally will increase blood flow to the arm, which results in a sudden sharp decrease 810 of the withdrawal pressure 802 from −150 mmHg to −390 mmHg at the occlusion detection event 811. The detection occurred when the withdrawal pressure 810 reached the withdrawal limit 803. The controller CPU responded by switching from the maximum flow control to the occlusion limit control for the duration of the partial occlusion 808. Flow control value was dynamically calculated from the occlusion pressure limit 803. That resulted in the overall reduction of blood flow to 25 to 45 mL/min following changing conditions in the circuit.

FIG. 8 illustrates the occlusion of the withdrawal line only. Although the infusion occlusion limit 801 is reduced in proportion to blood flow 804 during the occlusion period 808, the infusion line is never occluded. This can be determined by observing the occlusion pressure 809 always below the occlusion limit 801 by a significant margin, while the withdrawal occlusion limit 803 and the withdrawal pressure 802 intercept and are virtually equal during the period 808 because the PIFF controller is using the withdrawal occlusion limit 803 as a target.

The rapid response of the control algorithm is illustrated by immediate adjustment of flow in response to pressure change in the circuit. This response is possible due to: (a) servo controlled blood pump equipped with a sophisticated local DSP (digital signal processing) controller with high bandwidth, and (b) extremely low compliance of the blood path. The effectiveness of controls is illustrated by the return of the system to the steady state after the occlusion and or flow reduction disappeared at the point 812. Blood flow was never interrupted, alarm and operator intervention were avoided and the partial occlusion was prevented from escalation into a total occlusion (collapse of the vein) that would have occurred if not for the responsive control based on the withdrawal pressure.

If the system response was not this fast, it is likely that the pump would have continued for some time at the high flow of 55 mL/min. This high flow would have rapidly resulted in total emptying of the vein and caused a much more severe total occlusion. The failure to quickly recover from the total occlusion can result in the treatment time loss, potential alarms emitted from the extracorporeal system, and a potential need to stop treatment altogether, and/or undesired user intervention. Since user intervention can take considerable time, the blood will be stagnant in the circuit for a while. Stagnant blood can be expected to clot over several minutes and make the expensive circuit unusable for further treatment.

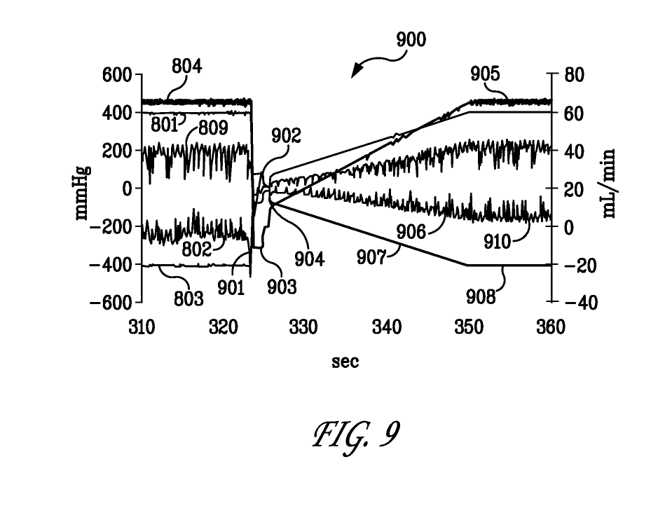

FIG. 9 illustrates a total occlusion of the blood withdrawal vein access in a different patient, but using the same apparatus as used to obtain the data shown in FIG. 8. Traces on the graph 900 are similar to those on the graph 800. The primary Y-axis (months) and secondary Y-axis (mL/min) correspond to pressure and flow, respectively, in the blood circuit. The X-axis is time in seconds. As in FIG. 8 the system is in steady state at the beginning of the graph. The blood flow 804 is controlled by the maximum flow algorithm and is equal to 66 mL/min. The withdrawal pressure 802 is at average of −250 mmHg and safely above the occlusion limit 803 at −400 mmHg until the occlusion event 901. Infusion pressure 809 is at average of 190 mmHg and way below the infusion occlusion limit 801 that is equal to 400 mmHg.

As depicted in FIG. 9, the occlusion of the withdrawal access is abrupt and total. The withdrawal vein has likely collapsed due to the vacuum generated by the needle or the needle opening could have sucked in the wall of the vein. The withdrawal vein is completely closed. Similar to the partial occlusion illustrated by FIG. 8, the rapid reduction of the blood flow 804 by the control system in response to the decreasing (more negative) withdrawal pressure 802 prevented escalation of the occlusion, but resulted in crossing of the occlusion limit 803 into positive values at the point 902. Simultaneously the blood flow 804 dropped to zero and sequentially became negative (reversed direction) for a short duration of time 903. The control system allowed reversed flow continued for 1 second at 10 mL/min as programmed into an algorithm. This resulted in possible re-infusion of 0.16 mL of blood back into the withdrawal vein. These parameters were set for the experiment and may not reflect an optimal combination. The objective of this maneuver is to release the vein wall if it was sucked against the needle orifice. It also facilitated the refilling of the vein if it was collapsed.

During the short period of time when the blood flow in the circuit was reversed, occlusion limits and algorithms in both infusion and withdrawal limbs of the circuit remained active. The polarity of the limits was reversed in response to the reversed direction of flow and corresponding pressure gradients.

The success of the maneuver is illustrated by the following recovery from total occlusion. At the point 904 signifying the end of allowed flow reversal, the withdrawal occlusion limit 803 became negative and the infusion occlusion limit 801 became positive again. The blood pump started the flow increase ramp shown between points 904 and 905. The gradual ramp at a maximum allowed rate is included in the total occlusion recovery algorithm to prevent immediate re-occlusion and to allow the withdrawal vein to refill with blood.

For the example illustrated by FIG. 9, the most likely cause of the occlusion was suction of the blood vessel wall to the withdrawal needle intake opening. The occlusion onset was rapid and the condition disappeared completely after the short reversal of flow that allowed the vessel to re-inflate. It can be observed that while the withdrawal occlusion ramp 907 followed the blood flow ramp 905, the measured withdrawal pressure 906 did not anymore intercept it. In fact, by the time the steady-state condition was restored, the withdrawal pressure 910 was at approximately −160 mmHg. Prior to occlusion the withdrawal pressure level 802 was approximately −200 mmHg. Thus, the withdrawal conditions have improved as a result of the total occlusion maneuver.

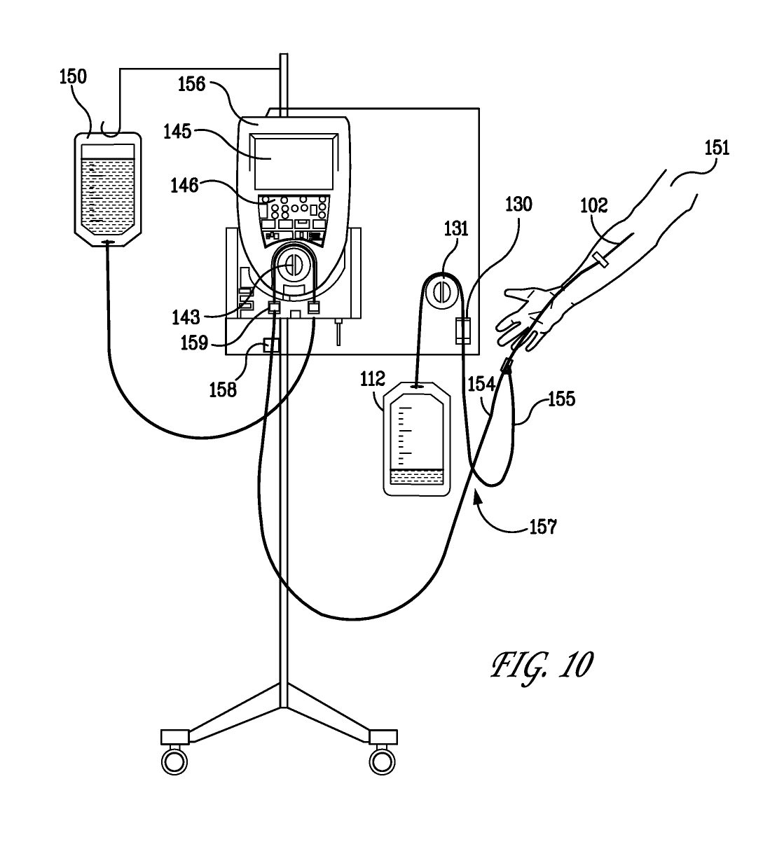

Another exemplary blood access apparatus described here is designed for the measurement of one or more characteristics of human blood, such as glucose concentration in blood. FIG. 10 illustrates the measurement of a blood characteristic using a measurement apparatus 156. The patient 151, such as a human or other mammal, can be treated while in bed or sitting in a chair and may be conscious or asleep. The apparatus can be attached to the patient in a doctor's office, an outpatient clinic, and can be suitable for use at home (provided that adequate supervision of a doctor or other medically trained person is present). The patient need not be confined to an intensive care unit (ICU), does not require surgery to be attached to the apparatus, and does not need specialized care or the continual presence of medical attendants.

A standard 18G (gage) or other appropriate catheter needle 102 can be introduced into a peripheral vein for the withdrawal and return of the blood. The procedure of inserting needles is similar to that used for inserting catheter needles to withdraw blood or for intravenous (IV) therapy. The needle is attached to withdrawal tubing 154 and flush tubing 155, respectively. The tubing can be secured to skin with adhesive tape.

The measurement apparatus includes a blood pump console 156 and a blood circuit 157. The console includes two rotating roller pumps that move blood and other fluids through the circuit, and the circuit is mounted to the console. The blood circuit includes withdrawal tubing 154 and the waste/flush tubing 155. The blood circuit includes a blood measurement system 158; pressure sensors 159 (in the withdrawal tube 154), and a saline source bag 150. The waste/flush line 157 is connected to pressure sensor 130, flush pump 131, and a waste bag 112. Blood passage through the circuit is preferably continuous during withdrawal, smooth and free of stagnate blood pools and air/blood interfaces. Continuous airless blood flow reduces the damping of pressure signals by the system and allows for a higher frequency response pressure controller, which allows the pressure controller to adjust the pump velocity more quickly to changes in pressure, thereby maintaining accurate pressure control without causing oscillation. The components of the circuit can be selected to provide smooth and continuous blood passages, such as with pressure sensors having cylindrical flow passages with electronic sensors embedded in a wall of the passage. The circuit can be provided in a sterile package for use for a single treatment. A more detailed description of an exemplary blood circuit is included in co-pending U.S. Pat. No. 6,887,214 which is incorporated by reference.

The circuit mounts with the blood pumps 143 and 131. The circuit can be mounted, primed and prepared for operation within minutes by one operator. The operator of the blood measurement apparatus, e.g., a nurse or medical technician, can set the maximum rate at which fluid is to be withdrawn and infused to the patient. These settings can be entered into the blood pump console 156 using the user interface, which can include a display 145 and control panel 146 with control keys for entering maximum flow rate and other controller settings. Information to assist the user in priming, setup and operation can be communicated to the operator, for example by display on an LCD (liquid crystal display) 145.

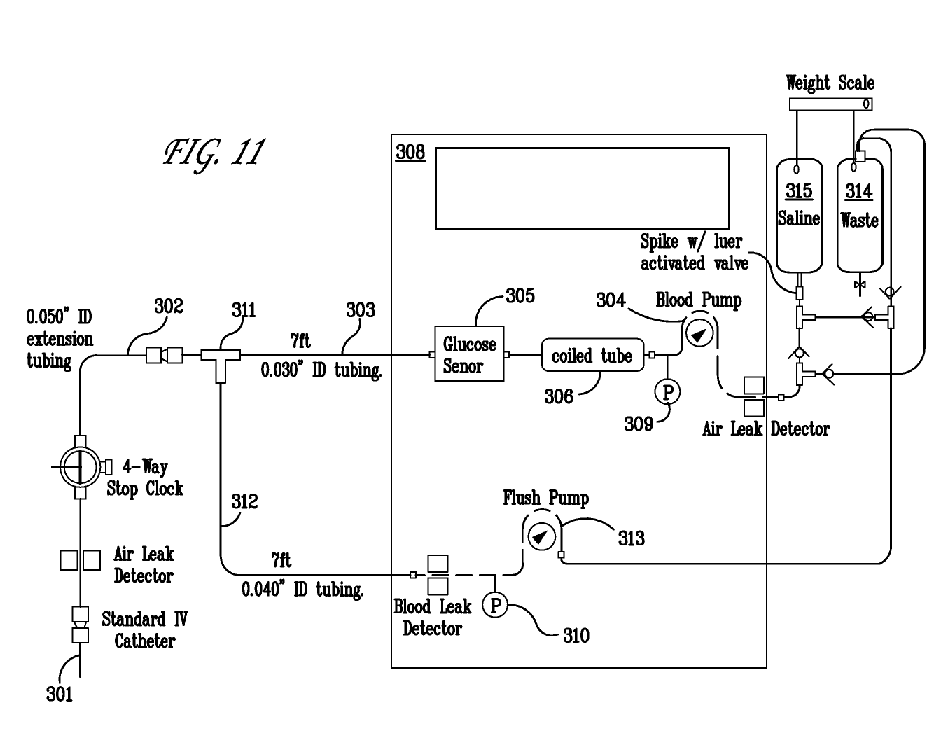

FIG. 11 illustrates the operation and fluid paths of blood, saline, and waste through a blood circuit such as that shown in FIG. 10. Blood is withdrawn from the patient through an 18 Gage or similar withdrawal needle 301. The withdrawal needle 301 can be inserted into a suitable peripheral vein in the patient's arm. The blood flow from the peripheral vein into the extension set 302 and withdrawal tubing 303 is dependent on the fluid pressure in that tubing which is controlled by a blood pump 304. Blood pump 304 can be operated in a constant flow mode with maximum pressure detection limits. Alternatively blood pump 304 can be operated in a constant pressure mode with or without maximum flow limits. As blood fills the withdrawal line the pressure needed to maintain constant flow increases. Therefore, when operated in a constant pressure mode the withdrawal rate is highest at the beginning of the draw and decreases thereafter. In operation, both blood pressure sensor 309 and flush/waste pressure sensor 310 can be used. During the withdrawal sequence pressure sensor 309 provides information on the pressure at the blood pump while pressure sensor 310 provides information on the pressure close to the patient. Pressure sensor 310 can therefore be used to effectively monitor the pressure at the patient and can be used for occlusion detection in the vein or extension set and inappropriate closure or partial closure of the stopcock. The pressure gradient between pressure sensor 310 and pressure sensor 309 can be used to assess flow characteristics of the withdrawal tubing. Factors that can influence this pressure differential include viscosity of the blood, the hematocrit of the blood, the presence of any occlusions, kinking of the tubing, compression of the tubing, or occlusions at the glucose sensor. The use of these two pressure sensors enables the blood access system to determine the location of any flow restrictions and any occlusions. As described previously the controller can compensate for these flow restrictions using a variety of compensating methodologies. Occlusion management methods can be similar or different based upon the location of the occlusion. For example, if the occlusion appears to be vein collapse at standard catheter 301 a reversal of flow followed by a pause period, and a slower withdrawal rate may be indicated. If the occlusion appears to be a kinking of the withdrawal line 303 an appropriate alarm can be sounded, while evidence of clot formation in the glucose sensor by necessitate a more vigorous cleaning cycle described later.

The blood withdrawal from standard catheter 301 to glucose sensor 305 can be done as a continuous withdrawal where the entire withdrawal tubing 303 is filled with blood. Other possibilities include the withdrawal of a blood segment large enough to enable an accurate measurement but less than having the entire withdrawal tube 303 filled with blood. If desired, an appropriately sized blood segment can be withdrawn past junction 311. At this point in the withdrawal sequence, flush pump 313 can be activated at a rate equal to, greater than, or less than the rate of blood pump 304. If the rate of flush pump 313 equals blood pump 304, the blood segment is transported to the glucose sensor with a following saline segment. If the rate of flush pump 313 exceeds the rate of blood pump 304 then there is a net fluid transfer towards glucose sensor 305 and towards standard catheter 301. This can help to clean the extension tubing while helping to push the blood towards glucose sensor 305. Pressure sensor 310 can be used to monitor any occlusions distal to junction 311 and can be used to determine when all of the blood has been returned to the patient. When flush pump 313 is operated at a rate equal to or greater than the rate of blood pump 304 the blood segment is “chased” by saline and can be referred to as a saline chaser. The use of this saline chaser following the blood segment can stabilize the pressure present at sensor 309. Keeping the pressure at sensor 309 within reasonable limits can become important when working with very high viscosity blood, typically associated with high hematocrit. Additionally, maintaining reasonable pumping pressures allows for lower power consumption, smaller motors and overall lower stresses on the pump components.

The blood withdrawn from the patient will mix with the saline in the withdrawal tubing to create a blood/saline mix at the boundary between the blood and saline. To ensure accurate blood measurements this blood/saline boundary should pass beyond the glucose sensor 305. This additional fluid volume can be stored in fluid reservoir 306. If needed for accurate blood glucose measurements, the blood can be moved in forward direction throughout the measurement cycle, can be moved back and forth over the glucose sensor, can be moved in a backward direction, or can be stationary during the glucose measurement.

Following completion of the glucose measurement the blood can be reinfused into the patient. Pressure sensor 310 can be used to detect occlusions between the junction 311 and the end of standard catheter 301. Additionally, any occlusions or flow problems between the blood pump and junction 311 can be assessed by examination of pressure measurements from sensor 309 or looking at a pressure differential between pressure sensor 309 and pressure sensor 310.

In one mode of operation, the blood access system can infuse the majority of blood back to the patient but seek to minimize overall saline infusion to the patient. To minimize saline infusion, some of the blood in the blood/saline mixture can be diverted to waste. In operation this diversion can occur as the blood/saline junction approaches or passes junction 311. At this point in circuit operation, flush pump 313 can be turned on at a rate comparable to blood pump 304. In this mode of operation, saline from a saline bag 315 is pumped through the withdrawal tubing 303 to junction 311 through flush tubing 312 and into waste bag 314. If there are flow restrictions or occlusions present in this circuit path pressure sensor 309 or pressure sensor 310 can be used to detect both the presence of the occlusion as well as the removal of the occlusion following cleaning.

In yet another operating mode utilizing pressure measurements, the system can be used to clean the tubing between the patient and junction 311. Such flushing or cleaning of the extension set can be accomplished by continuous, pulsed, forward and reverse, and turbulence generating flows. For the effective cleaning of Leuer connections, it has been found that higher, more turbulent flows can be advantageous. High flow rates, typically associated with high pressures, must be balanced against the risk of possible damage to the patient's blood vessel. For the purposes of creating maximal flow it can be advantageous to use both flush pump 313 and blood pump 314 to push saline through the extension tubing. In this mode of operation the use of both pressure sensors can ensure that vessel damage does not occur.

In addition to using pressure monitoring for occlusion in flow restriction detection and subsequent resolution, the pressure sensors can be used to determine disconnects. The circuit shown in FIG. 11 has a number of tubing connections that can fail and become disconnected. For example, if junction 311 becomes disconnected during operation, the pressures recorded at pressure sensors 310 would not be consistent with normal operation.

### EXAMPLE EMBODIMENTS

### Examples

The present invention can be realized in various embodiments, including without limitation those described following.

### Example 1

A method for controlling blood flow through an extracorporeal circuit coupled to a blood pump comprising: (a) withdrawing the blood from a blood vessel in a patient into the extracorporeal circuit, and measuring a characteristic of the blood in the circuit; (b) detecting an occlusion which at least partially blocks the withdrawal of blood from the patient, (c) interrupting step (a), and temporarily reversing the blood pump to reverse a flow of the blood to infuse blood from the circuit into the blood vessel after step (b), and (d) resuming step (a) after step (c).

### Example 2

A method for controlling blood flow as in example 1 wherein the occlusion is detected based on a pressure measurement of the blood in the circuit.

### Example 3

A method for controlling blood flow as in example 1 wherein blood flow is reversed after the blood flow is substantially reduced during step (a).

### Example 4

A method for controlling blood flow as in example 1 wherein blood flow is reversed after the blood flow is substantially reduced to zero flow during step (a).

### Example 5

A method for controlling blood flow as in example 1 wherein blood flow is reversed after the withdrawal blood vessel collapses.

### Example 6

A method for controlling blood flow as in example 1 wherein the flow is temporarily reversed after an occlusion has been detected in the withdrawal blood vessel.

### Example 7

A method for controlling blood flow as in example 1 wherein the blood flow is reversed for a predetermined duration.

### Example 8

A method for controlling blood flow as in example 1 wherein the reversed blood flow has a predetermined flow rate.

### Example 9

A method for controlling blood flow as in example 8 wherein the predetermined flow rate of the reversed blood flow is substantially less than the flow rate during step (a).

### Example 10

A method for controlling blood flow as in example 1 wherein the step (a) is resumed after step (c) when a flow capacity of the withdrawal blood vessel substantially increases.

### Example 11

A method for controlling blood flow as in example 1 further comprising gradually reducing blood flow prior to step (b).

### Example 12

A method of controlling an extracorporeal circuit having a blood pump comprising:

(a) withdrawing blood from a blood vessel in a patient into the extracorporeal circuit, and measuring a characteristic of the blood; (b) determining a withdrawal blood pressure in the extracorporeal circuit; (c) withdrawing blood at a flow rate based on an algorithm executed by the blood pump that correlates the flow rate to the withdrawal blood pressure; (d) interrupting step (a) to temporarily reverse the blood pump to reverse blood flow to infuse blood from the circuit into the blood vessel if the flow rate determined in step (c) is reduced to below a predetermined limit, and resuming step (a) after step (d).

### Example 13

A method of controlling an extracorporeal blood circuit as in example 12 wherein the predetermined limit is a blood flow rate of substantially zero.

### Example 14

A method of controlling an extracorporeal blood circuit as in example 12 further comprising: (e) detecting an occlusion which at least partially blocks the withdrawal of blood from the patient, and (f) temporarily reversing a flow of the blood to infuse blood from the circuit into the blood vessel if a pump controller executing an algorithm of withdrawal pressure as a function of desired flow and known resistance is unsuccessful in maintaining significant blood flow.

### Example 15

A method for controlling withdrawal of blood from a patient into an extracorporeal circuit which withdraws blood from a patient, measures a characteristic of the blood, and allows for detection of and recovery from a reduced flow capacity or total occlusion of a blood vessel, comprising: (a) reducing blood flow being withdrawn from the patient when a withdrawal pressure of the blood in the circuit becomes more negative than an occlusion limit that is a function of blood flow through the circuit, and (b) if the reduced blood flow is reduced below a predetermined minimal flow during step (a), then interrupting the withdrawal of blood, to temporarily reverse the blood pump to reverse the flow of blood in the circuit and infusing blood into the blood vessel.

### Example 16

A method as in example 15 further comprising: (c) prior to step (a), increasing a rate of blood being withdrawn until the blood vessel begins to collapse and occlude blood withdrawal; (d) prior to step (a), determining an occlusion withdrawal pressure corresponding to collapse of the vessel, e. initiating step (b) when the collapse withdrawal pressure is detected.

### Example 17

A method as in the example 16 wherein the collapse withdrawal pressure is a variable and a function of withdrawal blood flow.

### Example 18

A method as in example 16 wherein the collapse withdrawal pressure is periodically reestablished by stopping the blood flow through the circuit pump and then performing steps (c), (d) and (e).

### Example 19

A system for controlling blood flow withdrawn from a patient comprising: an extracorporeal circuit having a blood passage including a blood withdrawal tube and a blood measurement device; a withdrawal pressure sensor sensing a blood pressure in the withdrawal tube; a pump coupled to the circuit and adapted to move blood through the circuit at a controlled flow rate, and a pump controller receiving a blood pressure signal from the withdrawal pressure sensor and using the pressure signal to control the pump to regulate the controlled flow rate, wherein the pump controller includes a processor and a memory storing a control algorithm of a withdrawal pressure target, said controller adjusts the controlled flow rate based on a difference between a withdrawal pressure sensed by the withdrawal pressure sensor and the withdrawal pressure target.

### Example 20

A system as in example 19 wherein the pump controller includes a proportional integral feed forward pressure controller.

### Example 21

A system as in example 19 wherein the measurement device is a device that measures glucose in blood.

### Example 22

A system as in example 19 wherein the measurement device is a device that optically measures glucose concentration in blood.

### Example 23

A system as in example 19 wherein the pressure sensor is a real time sensor providing real time pressure signals to the pump controller.

### Example 24

A system as in example 19 wherein the pump includes a direct DC drive motor.

### Example 25

A system as in example 24 wherein the drive motor is a brushless motor.

### Example 26

A system as in example 19 wherein the measurement device is a device that electrochemically measures glucose in blood.

### Example 27

A system as in example 19, wherein the controller reverses the controlled flow rate based on a difference between a withdrawal pressure sensed by the withdrawal pressure sensor and the withdrawal pressure target.

### Example 28

A system as in example 19, wherein the controller reduces the controlled flow rate based on a difference between a withdrawal pressure sensed by the withdrawal pressure sensor and the withdrawal pressure target.

### Example 29

A method for controlling blood flow in a closed loop extracorporeal blood measurement system where blood is withdrawn and returned into a blood vessel in the same body in the process of measurement of a blood characteristic comprising: a. withdrawing blood from a blood vessel of the patient at a controlled flow rate; b. measuring a withdrawal pressure of the withdrawn blood; c. infusing at least a portion of the withdrawn blood into the patient; e. measuring the infusion pressure of the blood; f. comparing the withdrawal pressure to a withdrawal pressure target, and comparing the infusion pressure to an infusion pressure target; g. adjusting the flow rate to reduce a difference between the withdrawal pressure and a withdrawal pressure target, if the infusion pressure is less than an infusion pressure target, and h. adjusting the flow rate to reduce a difference between the infusion pressure and an infusion pressure target, if the infusion pressure is greater than the infusion pressure target.

### Example 30

A method as in example 29 wherein step (h) includes applying a difference between the infusion pressure and the infusion pressure target as feedback to adjust the flow rate of the blood being withdrawn.

### Example 31

A method as in example 29 wherein the blood is withdrawn through a needle and infused through said needle.

### Example 32

A method as in 29 wherein blood is withdrawn from and infused into a peripheral vein of a patient.

### Example 33

A method for limiting pump pressure in an extracorporeal blood measurement circuit coupled to a pump driven by a direct DC drive motor, wherein the method comprises: (a) measuring current used by the motor, (b) limiting pressure in said blood circuit by controlling said current, and (c) controlling the current based on a difference between a measured blood pressure in the circuit and a pressure target.

### Example 34

A method for controlling blood infusion into a mammalian patient in an air-free extracorporeal blood measurement system comprising: (a) infusing blood from the circuit into a blood vessel of the patient at a controlled infusion flow rate; (b) measuring an infusion pressure of the blood being infused; (c) comparing the measured infusion pressure to a disconnect pressure target; (d) sensing for air in the blood being infused; (e) generating a disconnection alarm if both the measured infusion pressure is no greater than the disconnect pressure target and air is sensed in the blood being infused.

### Example 35

A method for controlling blood infusion as in example 34, further comprising the step of reducing a rate of infusing blood if the measured infusion pressure is greater than the disconnect pressure target and air is not sensed in the blood being infused.

### Example 36

A method as in example 1 wherein the extracorporeal blood measurement system includes a glucose sensor and the flow reversal includes reversing a flow of blood through the glucose sensor.

### Example 37

A method as in example 1 wherein the blood pump is a rotating roller pump, and reversing the blood pump includes reversing a rotational direction of the roller pump.

### Example 38

A method as in example 1 wherein the reversal of flow of blood includes reversing the flow of blood throughout the extracorporeal blood measurement system.

### Example 39

A method as in example 12 wherein the extracorporeal blood measurement system includes a glucose sensor and the flow reversal includes reversing a flow of blood through the glucose sensor.

### Example 40

A method as in example 12 wherein the blood pump is a rotating roller pump, and reversing the blood pump includes reversing a rotational direction of the roller pump.

### Example 41

A method as in example 12 wherein the reversal of flow of blood includes reversing the flow of blood throughout the extracorporeal blood measurement system.

### Example 42

A method as in example 15 wherein the extracorporeal blood measurement system includes a glucose sensor and the flow reversal includes reversing a flow of blood through the glucose sensor.

### Example 43

A method as in example 15 wherein the blood pump is a rotating roller pump, and reversing the blood pump includes reversing a rotational direction of the roller pump.

### Example 44

A method as in example 15 wherein the reversal of flow of blood includes reversing the flow of blood throughout the extracorporeal blood measurement system.

### Example 45

A system as in example 19 wherein the extracorporeal circuit includes a glucose sensor, and the adjustment of the flow rate adjusts a flow of blood through the glucose sensor.

### Example 46

A system as in example 19 wherein the blood pump is a rotating roller pump, and reversing the blood pump includes reversing a rotational direction of the roller pump.

### Example 47

A method for automatically controlling blood flow through an extracorporeal blood measurement system coupled to a blood pump and a controller, said method comprising: a. withdrawing the blood from a withdrawal blood vessel in a patient into the extracorporeal blood measurement system; b. the controller automatically detecting an occlusion which at least partially blocks the withdrawal of blood from the patient; c. the controller automatically and temporarily ceasing the blood pump to temporarily cease a flow of the blood being withdraw after step (b), and d. the controller automatically resuming the pump and the flow of blood being withdrawn after a predetermined period of time.

### Example 48

A method for controlling blood flow as in example 47 wherein the occlusion is detected based on a pressure measurement of the blood flowing in the blood measurement system and made by the controller based on a pressure sensor in the blood circuit.

### Example 49

A method for controlling blood flow as in example 47 wherein blood flow is temporarily ceased after the blood flow is substantially reduced during step (a).

### Example 50

A method for controlling blood flow as in example 47 wherein the blood flow is temporarily ceased after the withdrawal blood vessel collapses.

### Example 51

A method for controlling blood flow as in example 47 wherein the flow is temporarily ceased after an occlusion has been detected in the withdrawal blood vessel.

### Example 52

A method for controlling blood flow as in example 47 wherein the blood flow is reversed for a predetermined duration of no more than two seconds.

### Example 53

A method for controlling blood flow as in example 47 further comprising gradually reducing blood flow prior to step (b).

### Example 54

A method of controlling an extracorporeal blood measurement system having a blood pump and a pump controller comprising: (a) withdrawing blood from a withdrawal blood vessel in a patient into the extracorporeal blood measurement system; (b) determining a withdrawal blood pressure in the extracorporeal blood measurement system; (c) withdrawing blood at a flow rate determined by the blood pump controller; (d) the pump controller automatically and temporarily ceasing the blood pump to cease the withdrawal of blood from the withdrawal blood vessel if the pressure determined in step (b) reduces to below a predetermined limit, and (e) the pump controller automatically restarting the blood pump to resume the withdrawal of blood from the withdrawal blood vessel.

The particular sizes and equipment discussed above are cited merely to illustrate particular embodiments of the invention. Additionally one of ordinary skill in the art can use a variety of circuits with pressure monitoring for improved operation through pending occlusion detection, and complete occlusion. Additionally, the use of two pressure monitors provides the ability to determine the location of a flow restriction and provide opportunities for correction. It is contemplated that the use of the invention can involve components having different sizes and characteristics. It is intended that the scope of the invention be defined by the claims appended hereto.

What is claimed is:

1. A method for controlling blood flow through an extracorporeal circuit including a glucose sensor and sensing glucose in blood in the extracorporeal circuit, wherein the circuit is coupled to a blood pump comprising:
(a) withdrawing the blood from a blood vessel in a patient into the extracorporeal circuit and measuring a characteristic of the blood in the circuit where the characteristic is glucose presence or concentration in the blood;
(b) detecting an occlusion which at least partially blocks the withdrawal of blood from the patient,
(c) interrupting step (a), and temporarily reversing the blood pump to reverse a flow of the blood to infuse blood from the circuit into the blood vessel after step (b), and
(d) resuming step (a) after step (c).

2. A method for controlling blood flow and sensing glucose as in claim 1 wherein the occlusion is detected based on a pressure measurement of the blood in the circuit.

3. A method for controlling blood flow and sensing glucose as in claim 1 wherein blood flow is reversed after the blood flow is substantially reduced during step (a).

4. A method for controlling blood flow and sensing glucose as in claim 1 wherein the flow reversal includes reversing a flow of blood through the glucose sensor.

5. A method for controlling blood flow and sensing glucose as in claim 1 wherein blood flow is reversed after the withdrawal blood vessel collapses.

6. A method for controlling blood flow and sensing glucose as in claim 1 wherein the flow is temporarily reversed after an occlusion has been detected in the withdrawal blood vessel.

7. A method for controlling blood flow and sensing glucose as in claim 1 wherein the blood flow is reversed for a predetermined duration.

8. A method for controlling blood flow and sensing glucose as in claim 1 wherein the reversed blood flow has a predetermined flow rate.

9. A method for controlling blood flow and sensing glucose as in claim 8 wherein the predetermined flow rate of the reversed blood flow is substantially less than the flow rate during step (a).

10. A method for controlling blood flow and sensing glucose as in claim 1 wherein the step (a) is resumed after step (c) when a flow capacity of the withdrawal blood vessel substantially increases.

11. A method for controlling blood flow and sensing glucose as in claim 1 further comprising gradually reducing blood flow prior to step (b).

12. A method of controlling an extracorporeal circuit having a blood pump and a glucose sensor and sensing glucose in the extracorporeal circuit, comprising:
(a) withdrawing blood from a blood vessel in a patient into the extracorporeal circuit, and measuring a characteristic of the blood using the glucose sensor, where the characteristic is glucose presence or concentration;
(b) determining a withdrawal blood pressure in the extracorporeal circuit;
(c) withdrawing blood at a flow rate based on an algorithm executed by the blood pump that correlates the flow rate to the withdrawal blood pressure;
(d) interrupting step (a) to temporarily reverse the blood pump to reverse blood flow to infuse blood from the circuit into the blood vessel if the flow rate determined in step (c) is reduced to below a predetermined limit, and resuming step (a) after step (d).

13. A method of controlling an extracorporeal blood circuit and sensing glucose as in claim 12 wherein the flow reversal includes reversing a flow of blood through the glucose sensor.

14. A method of controlling an extracorporeal blood circuit and sensing glucose as in claim 12 further comprising:
(e) detecting an occlusion which at least partially blocks the withdrawal of blood from the patient, and
(f) temporarily reversing a flow of the blood to infuse blood from the circuit into the blood vessel if a pump controller executing an algorithm of withdrawal pressure as a function of desired flow and known resistance is unsuccessful in maintaining significant blood flow.

15. A method for controlling withdrawal of blood from a patient into an extracorporeal circuit which withdraws blood from a patient, measures or detects one or more analytes in the blood, and allows for detection of and recovery from a reduced flow capacity or total occlusion of a blood vessel, comprising:
(a) measuring or detecting the one or more analytes in the blood;
(b) reducing blood flow being withdrawn from the patient when a withdrawal pressure of the blood in the circuit becomes more negative than an occlusion limit that is a function of blood flow through the circuit, and
(c) if the reduced blood flow is reduced below a predetermined minimal flow during step (b), then interrupting the withdrawal of blood, to temporarily reverse the blood pump to reverse the flow of blood in the circuit and infusing blood into the blood vessel.

16. A method as in claim 15 wherein the extracorporeal blood measurement system includes a glucose sensor.

17. A method as in claim 15 further comprising:
(c) prior to step (a), increasing a rate of blood being withdrawn until the blood vessel begins to collapse and occlude blood withdrawal;
(d) prior to step (a), determining an occlusion withdrawal pressure corresponding to collapse of the vessel, e. initiating step (b) when the collapse withdrawal pressure is detected.

18. A method as in the claim 17 wherein the collapse withdrawal pressure is a variable and a function of withdrawal blood flow.

19. A method as in claim 17 wherein the collapse withdrawal pressure is periodically reestablished by stopping the blood flow through the circuit pump and then performing steps (c), (d) and (e).

20. A system for controlling blood flow withdrawn from a patient and measuring or detecting analytes in the withdrawn blood, comprising:
a. an extracorporeal circuit having a blood passage including a blood withdrawal tube and a measurement device, wherein the measurement device measures or detects one or more of the analytes present in blood in the extracorporeal circuit;
b. a withdrawal pressure sensor sensing a blood pressure in the withdrawal tube;
c. a pump coupled to the circuit and adapted to move blood through the circuit at a controlled flow rates; and
d. a pump controller receiving a blood pressure signal from the withdrawal pressure sensor and using the pressure signal to control the pump to regulate the controlled flow rate, wherein the pump controller includes a processor and a memory storing a control algorithm of a withdrawal pressure target, said controller adjusts the controlled flow rate based on a difference between a withdrawal pressure sensed by the withdrawal pressure sensor and the withdrawal pressure target.

21. A system as in claim 20 wherein the measurement device comprises a glucose sensor.

22. A system as in claim 20 wherein the pump controller includes a proportional integral feed forward pressure controller.

23. A system as in claim 20 wherein the measurement device is a device that measures glucose in blood.

24. A system as in claim 20 wherein the measurement device is a device that optically measures glucose concentration in blood.

25. A system as in claim 20 wherein the measurement device is a device that electrochemically measures glucose in blood.

26. A system as in claim 20, wherein the controller reverses the controlled flow rate based on a difference between a withdrawal pressure sensed by the withdrawal pressure sensor and the withdrawal pressure target.

27. A system as in claim 20, wherein the controller reduces the controlled flow rate based on a difference between a withdrawal pressure sensed by the withdrawal pressure sensor and the withdrawal pressure target.

28. A method for controlling blood flow in a closed loop extracorporeal blood measurement system and measuring or detecting one or more analytes in the blood, where blood is withdrawn and returned into a blood vessel in the same body in the process of measurement of a blood characteristic comprising:
a. withdrawing blood from a blood vessel of the patient at a controlled flow rate;
b. measuring or detecting one or more analytes in the blood;
c. measuring a withdrawal pressure of the withdrawn blood;
d. infusing at least a portion of the withdrawn blood into the patient;
e. measuring the infusion pressure of the blood;
f. comparing the withdrawal pressure to a withdrawal pressure target, and comparing the infusion pressure to an infusion pressure target;
g. adjusting the flow rate to reduce a difference between the withdrawal pressure and a withdrawal pressure target, if the infusion pressure is less than an infusion pressure target, and
h. adjusting the flow rate to reduce a difference between the infusion pressure and an infusion pressure target, if the infusion pressure is greater than the infusion pressure target.

29. A method as in claim 28 wherein step (h) includes applying a difference between the infusion pressure and the infusion pressure target as feedback to adjust the flow rate of the blood being withdrawn.

30. A method as in claim 28 wherein the blood is withdrawn through a needle and infused through said needle.

31. A method as in 28 wherein blood is withdrawn from and infused into a peripheral vein of a patient.
